(19) United States
(12) Patent Application Publication
(10) Pub. No.: US 2024/0189341 A1
(43) Pub. Date: Jun. 13, 2024

(54) PHARMACEUTICAL COMBINATION AND USE THEREOF

(71) Applicant: REMEGEN CO., LTD., Yantai (CN)

(72) Inventors: Xinling ZHANG, Yantai (CN); Wenting LUO, Yantai (CN); Yue WANG, Yantai (CN); Jie ZHOU, Yantai (CN); Qilin YIN, Yantai (CN); Changjiang HUANG, Yantai (CN); MARIE M. ZHU, Yantai (CN)

(21) Appl. No.: 18/011,194

(22) PCT Filed: Jun. 21, 2022
(86) PCT No.: PCT/CN2022/100012

§ 371 Date: Dec. 19, 2022

(30) Foreign Application Priority Data
Jun. 22, 2021 (CN) 202110689544.3

(51) Int. Cl. A61K 31/724; A61K 47/68; A61P 35/00; C07K 16/32

(52) U.S. Cl. CPC A61K 31/724; A61K 47/6877; A61P 35/00; C07K 16/32; C07K 2317/24

(57) ABSTRACT

The present disclosure provides a pharmaceutical combination and use thereof. By using an effective amount of methyl-β-cyclodextrin as a preparation component or a combined auxiliary drug, the efficacy of ADC can be significantly improved, so that some of the ADC drugs with safety issues caused by excessive dosage can be developed. Moreover, due to the reduction in the dosage of ADC drugs, the cost of production and the cost of treatment for patients have also been greatly reduced, thereby being beneficial.

### CROSS-REFERENCE OF RELATED APPLICATIONS

The present application is the national phase of International Application No. PCT/CN2022/100012, titled “PHARMACEUTICAL COMBINATION AND USE THEREOF”, filed on Jun. 21, 2022, which claims priority to Chinese Patent Application No. 202110689544.3, titled “PHARMACEUTICAL COMBINATION AND USE THEREOF”, filed on Jun. 22, 2021 with the China National Intellectual Property Administration, which are incorporated herein by reference in their entireties.

### FIELD

The present disclosure relates to the field of biomedicine, in particular to use of methyl-β-cyclodextrin in ADC drug preparation.

### BACKGROUND

In recent years, the overall incidence of malignant tumors in the world has shown a continuously increasing trend, which is a serious threat to human health and survival. Currently, surgery, chemotherapy and radiotherapy are mainly used in clinical treatment of malignant tumors, but it is difficult to achieve satisfactory curative effect. Antibody-drug conjugate (ADC) is a kind of biomedicine, in which biologically active cytotoxin (drug) links with an antibody through a chemical linker. After the toxin and antibody are conjugated, the antibody-drug conjugate specifically recognizes and binds to the receptor on the surface of the cancer cell by using the targeting ability of monoclonal antibody, then enters the cell through endocytosis, and releases cytotoxin by using the protease in the cell to prevent cancer cells from reproduction and kill cancer cells. In the prior art, mammalian cell culture is generally used to produce and express antibodies, and the highly purified antibody is conjugated with the cytotoxin MMAE through a linker to obtain an antibody-drug conjugate. Antibody-drug conjugation technology integrates small molecule toxin drugs and biological proteins, and has the advantages of both, which becomes a new generation of therapeutic products, greatly enhancing drug efficacy and simultaneously reducing toxic side effects.

At present, ADC has made breakthroughs in the treatment of malignant tumors, which becomes a major emerging treatment method after surgery, chemotherapy and radiotherapy. However, as of June 2021, only 12 ADCs have been approved globally (10 by the US FDA, one by Japan PMDA, and one by China).

TABLE 1
Marketed antibody-drug conjugates

| Generic name of drug | Company | Target | Time to market |
|---|---|---|---|
| Brentuximab vedotin | Seattle, Takeda | CD30 | Marketed/2011 |
| Ado-trastuzumab | Genentech | Her2 | Marketed/2013 |
| emtansine | | | |
| Inotuzumab | Pfizer | CD22 | Marketed/2017 |
| ozogamicin | | | |
| Gemtuzumab | Pfizer | CD33 | Marketed/2017 |
| ozogamicin | | | |
| Moxetumomab | AstraZeneca | CD22 | Marketed/2018 |
| pasudotox-tdfk | | | |
| Enfortumab | Seattle/Astellas | Nectin-4 | Marketed/2019 |
| vedotin-ejfv | | | |
| Polatuzumab | Genentech | CD79b | Marketed/2019 |
| vedotin-piiq | | | |
| Fam-trastuzumab | AstraZeneca/Daiichi | Her2 | Marketed/2019 |
| deruxtecan-nxki | Sankyo | | [symbol] |
| Sacituzumab | Immunomedics | Trop2 | Marketed/2020 |
| govitecan-hziy | | | |
| Belantamab mafodotin | Glaxosmithkline | BCMA | Marketed/2020 |
| | (Ireland) Ltd | | |
| Cetuximab sarotalocan | Rakuten Medical | EGFR | Marketed/2020 |
| Disitamab Vedotin | Remegen Co., Ltd. | Her2 | Marketed/2021 |
| | (Yantai) | | |

Note:
Mylotarg was withdrawn from the market in 2010 after approved for marketing in 2000, and was re-approved for marketing in 2017.

The reason why there are so few ADCs approved is mainly due to the problems of conjugating technology, targeting ability, effectiveness, safety and the like of ADC drug preparations. In all failed cases, drug efficacy and safety are the most important reasons, whose proportions are as high as 52% and 24%. Improving the effect of ADC drugs through formulation components or combined auxiliary drugs is currently an exploration approach.

Methyl-β-cyclodextrin (CAS No. 128446-36-6) is a macrocyclic compound with a molecular formula of C₅₄H₉₄O₃₅ and a molecular weight of 1303.3, which can form inclusion complexes with many guest molecules. Compared with its parent β-cyclodextrin, it has higher solubility in aqueous solution and higher solubilizing and complexing ability. Moreover, it can also increase the solubility of non-polar substances, such as fatty acids, lipids, vitamins and cholesterol, which can be used in cell culture.

### SUMMARY

The present disclosure surprisingly found that, by using an effective amount of methyl-β-cyclodextrin, the dosage of ADC drug can be reduced, and the safety of ADC drug can be further guaranteed while the therapeutic effect is ensured.

Specifically, the present disclosure provides use of an effective amount of methyl-β-cyclodextrin in reducing the dosage of an antibody-drug conjugate drug in treatment. Wherein, the use refers to the combined application of an effective amount of methyl-β-cyclodextrin and an antibody-drug conjugate preparation, or methyl-β-cyclodextrin as an excipient component of an antibody-drug conjugate drug preparation.

Further, the molar ratio of methyl-β-cyclodextrin to the antibody-drug conjugate in the drug is 100˜60000:0.001˜100; or 200˜50000:0.001˜50; or 200˜40000:0.01˜50; or 200˜40000:0.0120; or 200˜40000:0.01˜10; preferably, the molar ratio is 250˜39000:0.01˜1; or 200˜39000:0.01˜0.5; more preferably, the molar ratio is 300˜38170:0.02˜0.2.

Further, the antibody-drug conjugate is used for the treatment of tumors, autoimmune diseases or infectious diseases.

In some specific embodiments, the target of the antibody-drug conjugate is selected from BCMA, CD79B, c-Met, GPNMB, IL2RA, LY6E, CD1, CD1a, CD2, CD3, CD4, CD5, CD8, CD11A, CD14, CD15, CD16, CD18, CD19, CD20, CD21, CD22, CD23, CD25, CD29, CD30, CD32b, CD33, CD37, CD38, CD40, CD40L, CD44, CD45, CD46, CD52, CD54, CD55, CD59, CD64, CD67, CD70, CD74, CD79a, CD79b, CD80, CD83, CD95, CD126, CD133, CD138, CD147, CD154, CD166, CD276, HER1, HER2, HER3, MUC1, PTK7, STEAP1, VTCN1, AXL, BCMA, CA9, CASP, CASP3, CDH3, CDKs, CEACAM5, CLDN18, c-Met, Cripto-1, CTL4, DLL3, EF2, EFNA4, EGFR, ENPP3, EphA2, ETBR, FGFR2, FGFR3, FOLR1, FOLR1, Ganglioside, GCPII, HER2, HER3, HGFR, HLA-DR, IGF1R, IL3RA, ITGAV, ITGB3, KIT, LAMP1, Lewis-Y, LRRC15, LY75, LYPD3, MCP, MELTF, MSLN, MUC1, MUC16, NaPi-2b, NCAM1, NECTIN4, NOTCH3, Prolactin receptor, RNA polymerase II, ROR1, SDC1, SGLT2, SLAMF6, SLAMF7, SLITRK6, STAR, STING, TfR, TIM1, TLR8, TNF, TOP1, TPBG, Trop-2, VEGF, ZIP6, cytokines, tubulins and combinations thereof;

In some more specific embodiments, the target of the antibody-drug conjugate is selected from CD19, EGFR, BCMA, Trop-2, TOP1, NECTIN4, CD79B, CD22, HER2, CD30, CD33, c-Met, cytokines, tubulins and combinations thereof.

In some embodiments, the antibody-drug conjugate is selected from Loncastuximab tesirine, Cetuximab sarotalocan, Belantamab mafodotin, Sacituzumab govitecan, Fam-trastuzumab deruxtecan, Enfortumab vedotin, Polatuzumab vedotin, Inotuzumab ozogamicin, Ado-trastuzumab emtansine, Brentuximab vedotin, Gemtuzumab ozogamicin, Disitamab vedotin, Tisotumab vedotin, Depatuxizumab mafodotin, TAA-013, Trastuzumab duocarmazine, KSI-301, BAT-8001, Rovalpituzumab tesirine, SAR-408701, datopotamab, Mirvetuximab soravtansine, ARX-788, Trastuzumab emtansine, Telisotuzumab vedotin, SHR-A1403. Wherein:

| Drug name | Original research company |
|---|---|
| Loncastuximab tesirine | Adc Therapeutics; |
| Cetuximab sarotalocan | Aspyrian Therapeutics; |
| Belantamab mafodotin | GlaxoSmithKline; |
| Sacituzumab govitecan | Immunomedics Inc; |
| Fam-trastuzumab deruxtecan | Daiichi Sankyo; |
| Enfortumab vedotin | Agensys; Seattle Genetics; |
| Polatuzumab vedotin | Seattle Genetics; |
| | Genentech; |
| Inotuzumab Ozogamicin | Pfizer; Belgian UCB |
| | Pharmaceuticals Ltd.; |
| Ado-trastuzumab emtansine | Genentech; |
| Brentuximab vedotin | Seattle Genetics Inc; Millennium |
| | Pharmaceuticals Inc; |
| Gemtuzumab ozogamicin | Pfizer; Belgian UCB |
| | Pharmaceuticals Ltd.; |
| Disitamab Vedotin | Remegen Co., Ltd. (Yantai); |
| Tisotumab vedotin | Genmab; |
| Depatuxizumab mafodotin | Abbvie; |
| TAA-013 | TOT Biopharm Co., Ltd.; |
| Trastuzumab duocarmazine | Synthon; |
| KSI-301 | Kodiak Sciences; |
| BAT-8001 | Bio-Thera Solutions, Ltd.; |
| Rovalpituzumab tesirine | Abbvie; Stemcentrx; |
| SAR-408701 | Immunogen; |
| datopotamab | Daiichi Sankyo; |
| Mirvetuximab soravtansine | Immunogen Inc; |
| ARX-788 | Ambrx; |
| Trastuzumab emtansine | Zydus Cadila; |
| Telisotuzumab vedoti | Abbvie |
| SHR-A1403 | Jiangsu Hengrui Pharmaceuticals |
| | Co., Ltd. |

In other embodiments, the heavy chain variable region CDRs of the antibody or antigen-binding fragment of the antibody-drug conjugate are shown in SEQ ID NOs: 1-3 (Kabat number), the light chain variable region CDRs are shown in SEQ ID NOs: 4-6 (Kabat number); specifically, the amino acid sequences of the heavy chain variable regions and light chain variable regions thereof are shown in SEQ ID NOs: 7-8; more specifically , the amino acid sequences of the heavy chains and light chains thereof are shown in SEQ ID NOs: 9-10.

| SEQ ID NO: 1 (heavy chain variable region CDR1): | |
|---|---|
| SYWMH | |
| SEQ ID NO: 2 (heavy chain variable region CDR2): | |
| MIDPSDSESR LNQKFKD | |
| SEQ ID NO: 3 (heavy chain variable region CDR3): | |
| SGYHGTSYWY FDV | |
| SEQ ID NO: 4 (light chain variable region CDR1): | |
| RSSKSLLHSD GITYLY | |
| SEQ ID NO: 5 (light chain variable region CDR2): | |
| QMSNLAS | |
| SEQ ID NO: 6 (light chain variable region CDR3): | |
| AQNLELPPT | |
| SEQ ID NO: 7 (heavy chain variable region): | |
| QVQLEQSGPE VVKPGASVKV SCKASGYSFT SYWMHWVRQA PGQGLEWMGM IDPSDSESRL | 60 |
| NQKFKDRVTM TVDTPTSTVY MELSSPRSED TAVYYCARSG YHGTSYWYFD VWGQGTLVTV | 120 |
| SS | 122 |
| SEQ ID NO: 8 (light chain variable region): | |
| DIVMTQSPLS LPVTPGEPAS ISCRSSKSLL HSDGITYLYW YLQKPGQSPQ LLIYQMSNLA | 60 |
| SGVPDRFSGS GSGTDFTLKI SRVEAEDVGV YYCAQNLELP PTFGGGTKVE IKRTV | 115 |
| SEQ ID NO: 9 (heavy chain): | |
| QVQLEQSGPE VVKPGASVKV SCKASGYSFT SYWMHWVRQA PGQGLEWMGM IDPSDSESRL | 60 |
| NQKFKDRVTM TVDTPTSTVY MELSSPRSED TAVYYCARSG YHGTSYWYFD VWGQGTLVTV | 120 |
| SSASTKGPSV FPLAPSSKST SGGTAALGCL VKDYFPEPVT VSWNSGALTS GVHTFPAVLQ | 180 |
| SSGLYSLSSV VTVPSSSLGT QTYICNVNHK PSNTKVDKKV EPKSCDKTHT CPPCPAPELL | 240 |
| GGPSVFLFPP KPKDTLMISR TPEVTCVVVD VSHEDPEVKF NWYVDGVEVH NAKTKPREEQ | 300 |
| YNSTYRVVSV LTVLHQDWLN GKEYKCKVSN KALPAPIEKT ISKAKGQPRE PQVYTLPPSR | 360 |
| EEMTKNQVSL TCLVKGFYPS DIAVEWESNG QPENNYKTTP PVLDSDGSFF LYSKLTVDKS | 420 |
| RWQQGNVFSC SVMHEALHNH YTQKSLSLSP GK | 452 |
| SEQ ID NO: 10 (light chain): | |
| DIVMTQSPLS LPVTPGEPAS ISCRSSKSLL HSDGITYLYW YLQKPGQSPQ LLIYQMSNLA | 60 |
| SGVPDRFSGS GSGTDFTLKI SRVEAEDVGV YYCAQNLELP PTFGGGTKVE IKRTVAAPSV | 120 |
| FIFPPSDEQL KSGTASVVCL LNNFYPREAK VQWKVDNALQ SGNSQESVTE QDSKDSTYSL | 180 |
| SSTLTLSKAD YEKHKVYACE VTHQGLSSPV TKSENRGEC | 219 |

In other specific embodiments, the antibody-drug conjugate has the following structure:

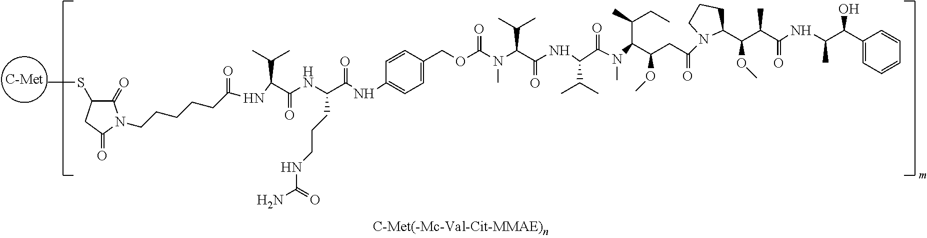

wherein: m represents an integer selected from 1, 2, 3, 4, 5, 6, 7, and 8; “C-Met” represents an antibody targeting C-Met, and in some preferred embodiments, the antibody targeting C-Met is a monoclonal antibody or a functional fragment thereof. In some specific embodiments, the C-Met antibody has the CDR sequences of the heavy chain variable region as described in SEQ ID NOs: 1-3, and/or the CDR sequences of the light chain variable region as described in SEQ ID NO: 4-6. In some more specific embodiments, the C-Met antibody has the amino acid sequence of the heavy chain variable region as described in SEQ ID NO: 7 and/or the amino acid sequence of the light chain variable region as described in SEQ ID NO: 8. In other further more specific embodiments, the C-Met antibody has the amino acid sequence of the heavy chain as described in SEQ ID NO: 9, and/or the amino acid sequence of the light chain as described in SEQ ID NO: 10.

In some specific embodiments, the antibody-drug conjugate has the following structure:

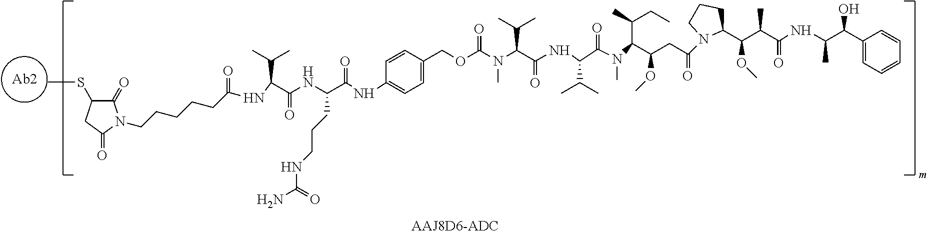

The heavy chain variable region CDR sequence of the Ab2 is shown in SEQ ID NO: 1-3, the light chain variable region sequence CDR sequence is shown in SEQ ID NO: 4-6, the heavy chain variable region sequence is shown in SEQ ID NO: 7, the light chain variable region sequence is shown in SEQ ID NO: 8, the heavy chain amino acid sequence is shown in SEQ ID NO: 9, and the light chain amino acid sequence is shown in SEQ ID NO: 10; and the average DAR value thereof is 4.02.

In other embodiments, the heavy chain variable region CDRs of the antibody or antigen-binding fragment of the antibody-drug conjugate are shown in SEQ ID NOs: 11-13 (IMGT number), the light chain variable region CDRs are shown in SEQ ID NOs: 14-16 (IMGT number); specifically, the amino acid sequences of the heavy chain variable region and light chain variable region thereof are shown in SEQ ID NOs: 17-18; more specifically, the amino acid sequences of the heavy chain and light chain thereof are shown in SEQ ID NOs: 19-20.

| SEQ ID NO: 11 (heavy chain variable region CDR1): | |
|---|---|
| GYTFTDYY | |
| SEQ ID NO: 12 (heavy chain variable region CDR2): | |
| VNPDHGDS | |
| SEQ ID NO: 13 (heavy chain variable region CDR3): | |
| ARNYLFDH | |
| SEQ ID NO: 14 (light chain variable region CDR1): | |
| QDVGTA | |
| SEQ ID NO: 15 (light chain variable region CDR2): | |
| WAS | |
| SEQ ID NO: 16 (light chain variable region CDR3): | |
| HQFATYT | |
| SEQ ID NO: 17 (heavy chain variable region): | |
| EVQLVQSGAE VKKPGATVKI SCKVSGYTFT DYYIHWVQQA PGKGLEWMGR VNPDHGDSYY | 60 |
| NQKFKDKATI TADKSTDTAY MELSSLRSED TAVYFCARNY LFDHWGQGTL VTVSS | 115 |
| SEQ ID NO: 18 (light chain variable region): | |
| DIQMTQSPSS VSASVGDRVT ITCKASQDVG TAVAWYQQKP GKAPKLLIYW ASIRHTGVPS | 60 |
| RFSGSGSGTD FTLTISSLQP EDFATYYCHQ FATYTFGGGT KVEIKRTV | 108 |
| SEQ ID NO: 19 (heavy chain): | |
| EVQLVQSGAE VKKPGATVKI SCKVSGYTFT DYYIHWVQQA PGKGLEWMGR VNPDHGDSYY | 60 |
| NQKFKDKATI TADKSTDTAY MELSSLRSED TAVYFCARNY LFDHWGQGTL VTVSSASTKG | 120 |
| PSVFPLAPSS KSTSGGTAAL GCLVKDYFPE PVTVSWNSGA LTSGVHTFPA VLQSSGLYSL | 180 |
| SSVVTVPSSS LGTQTYICNV NHKPSNTKVD KKVEPKSCDK THTCPPCPAP ELLGGPSVFL | 240 |
| FPPKPKDTLM ISRTPEVTCV VVDVSHEDPE VKENWYVDGV EVHNAKTKPR EEQYNSTYRV | 300 |
| VSVLTVLHQD WLNGKEYKCK VSNKALPAPI EKTISKAKGQ PREPQVYTLP PSREEMTKNQ | 360 |
| VSLTCLVKGF YPSDIAVEWE SNGQPENNYK TTPPVLDSDG SFFLYSKLTV DKSRWQQGNV | 420 |
| FSCSVMHEAL HNHYTQKSLS LSPGK | 445 |
| SEQ ID NO: 20 (light chain): | |
| DIQMTQSPSS VSASVGDRVT ITCKASQDVG TAVAWYQQKP GKAPKLLIYW ASIRHTGVPS | 60 |
| RFSGSGSGTD FTLTISSLQP EDFATYYCHQ FATYTFGGGT KVEIKRTVAA PSVFIFPPSD | 120 |
| EQLKSGTASV VCLLNNFYPR EAKVQWKVDN ALQSGNSQES VTEQDSKDST YSLSSTLTLS | 180 |
| KADYEKHKVY ACEVTHQGLS SPVTKSENRG EC | 212 |

In some specific embodiments, the antibody-drug conjugate has the following structure:

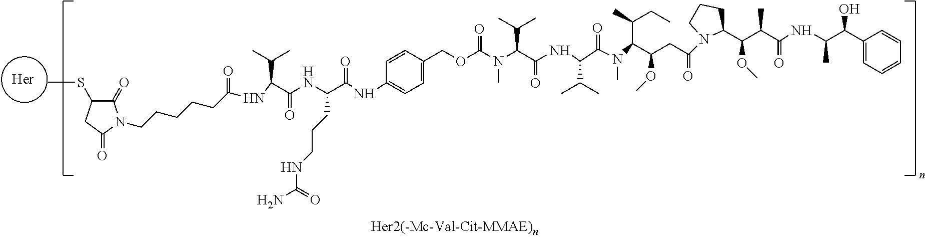

wherein: n represents an integer selected from 1, 2, 3, 4, 5, 6, 7, 8; “Her2” represents an antibody targeting Her2, and in some preferred embodiments, the antibody targeting to Her2 is a monoclonal antibody or a functional fragment thereof. In some specific embodiments, the Her2 antibody has the CDR sequences of the heavy chain variable region as described in SEQ ID NOs: 11-13, and/or has the CDR sequences of the light chain variable region as described in SEQ ID NOs: 14-16. In some more specific embodiments, the Her2 antibody has the amino acid sequence of heavy chain variable region as described in SEQ ID NO: 17 and/or the amino acid sequence of light chain variable region as described in SEQ ID NO: 18. In other further more specific embodiments, the Her2 antibody has the heavy chain amino acid sequence as described in SEQ ID NO: 19 and/or the light chain amino acid sequence as described in SEQ ID NO: 20.

In some embodiments, the antibody-drug conjugate is Disitamab vedotin.

Further, the tumor is a solid tumor or a non-solid tumor; preferably, the tumor is selected from hematopoietic tumor, carcinoma, sarcoma, melanoma or glial tumor; more preferably, the tumor is selected from solid tumors or blood tumors such as breast cancer, ovarian cancer, cervical cancer, uterine cancer, prostate cancer, kidney cancer, urethral cancer, bladder cancer, liver cancer, stomach cancer, endometrial cancer, salivary gland cancer, esophagus cancer, lung cancer, colon cancer, rectal cancer, colorectal cancer, bone cancer, skin cancer, thyroid cancer, pancreatic cancer, melanoma, glioma, neuroblastoma, glioblastoma multiforme, sarcoma, lymphoma and leukemia.

Further, the autoimmune disease is non-limitingly selected from immune-mediated thrombocytopenia, dermatomyositis, Sjogren's syndrome, multiple sclerosis, Siddenham's chorea, myasthenia gravis, systemic lupus erythematosus, lupus nephritis, rheumatic fever, rheumatoid arthritis, polyglandular syndrome, bullous pemphigoid, diabetes, Hen-Scherer's purpura, post-streptococcal nephritis, erythema nodosum, Takayasu's arteritis, Addison's disease, sarcoidosis, ulcerative colitis, erythema multiforme, IgA nephropathy, polyarteritis nodosa, ankylosing spondylitis, Goodpas' Hill syndrome, thromboangiitis obliterans, primary biliary cirrhosis, Hashimoto's thyroiditis, thyrotoxicosis, scleroderma, chronic active hepatitis, polymyositis/dermatomyositis, polychondritis, pemphigus vulgaris, Wegener's granulomatosis, membranous nephropathy, amyotrophic lateral sclerosis, tabes, giant cell arteritis/polymyalgia, pernicious anemia, rapidly progressive glomerulonephritis, fibrotic alveolitis, and juvenile diabetes and emerging diseases.

Further, the infectious disease is non-limitingly selected from human immunodeficiency virus (HIV), Mycobacterium tuberculosis, Streptococcus agalactiae, Methicillin-resistant Staphylococcus aureus, Legionella pneumophila, Streptococcus pyogenes, Escherichia coli, Neisseria gonorrhoeae, Neisseria meningitidis, Pneumococcus, Haemophilus influenzae type B, Treponema pallidus, Lyme disease Treponema, West Nile virus, Pseudomonas aeruginosa, Mycobacterium leprae, Bacillus abortus, rabies virus, influenza virus, cytomegalovirus, herpes simplex virus type I, herpes simplex virus type II, human serum parvovirus, respiratory syncytial virus, varicella-zoster virus, Hepatitis B virus, measles virus, adenovirus, human T-cell leukemia virus, Epstein-Barr virus, murine leukemia virus, mumps virus, vesicular stomatitis virus, Sindbis virus, lymphocytic choriomeningitis virus, wart virus, bluetongue virus, Sendai virus, feline leukemia virus, reovirus, polio virus, simian virus 40, murine mammary tumor virus, dengue virus, rubella virus, Plasmodium falciparum, Plasmodium vivax, Toxoplasma gondii, Trypanosoma johnsonii, Trypanosoma cruzi, Trypanosoma rhodesiana, Trypanosoma brucei, Schistosoma mansonii, Schistosoma japonicum, Babesia bovis, Eimeria tenella, Onchocerciasis, tropical Leishmania, Trichinella spiralis, Pyremys vulgaris, Taenia vesicularis, Taenia lamblia, Taenia saginata, Echinococcus granulosus, Taenia mesogenes, Mycoplasma arthritis, Mycoplasma hyorrhis, Mycoplasma oralis, Mycoplasma pyogenes, Acholesteria reinhardtii, Mycoplasma salivarius and Mycoplasma pneumoniae and emerging diseases.

In the embodiments provided by the present disclosure, the methyl-β-cyclodextrin is used as one of the excipient components of an antibody-drug conjugate preparation.

In the embodiments provided by the present disclosure, the methyl-β-cyclodextrin is developed into a methyl-β-cyclodextrin preparation and used in combination with an antibody-drug conjugate drug.

The present disclosure also provides use of methyl-β-cyclodextrin in the preparation of a medicament for reducing the therapeutic dosage of an antibody-drug conjugate.

The present disclosure also provides an antibody-drug conjugate preparation, which includes an effective amount of methyl-β-cyclodextrin as excipient.

Further, the molar ratio of methyl-β-cyclodextrin to the antibody-drug conjugate is 100˜60000:0.001˜100; or 200˜50000:0.001˜50; or 200˜40000:0.01˜50; or 200˜40000:0.01˜20; or 200˜40000:0.01˜10; preferably, the molar ratio is 250˜39000:0.01˜1; or 200˜39000:0.01˜0.5; more preferably, the molar ratio is 290˜38360:0.02˜0.15.

The present disclosure also provides a method for treating diseases with a drug combination, wherein the drug combination includes an effective dosage of methyl-β-cyclodextrin or a pharmaceutically acceptable excipient thereof and an antibody-drug conjugate or a pharmaceutically acceptable excipient thereof; wherein, the disease is selected from tumors, autoimmune diseases and infectious diseases.

In some preferred embodiments, the molar ratio of methyl-β-cyclodextrin to the antibody-drug conjugate is 200˜40000:0.001˜100; preferably, the molar ratio is 250˜39000:0.01˜10; more preferably, the molar ratio is 290-38360:0.02-0.15.

The methyl-β-cyclodextrin and antibody-drug conjugate used in the present disclosure can be a liquid preparation or a freeze-dried preparation. When combined administration is applied, the administration may be simultaneous or sequential.

The present disclosure also provides use of a preparation formed by methyl-β-cyclodextrin or a pharmaceutically acceptable excipient thereof, and a preparation formed by an antibody-drug conjugate or a pharmaceutically acceptable excipient thereof in the preparation of a medicament for treating cancer, autoimmune diseases, and infectious diseases.

The methyl-β-cyclodextrin found in the present disclosure can significantly improve the efficacy of ADC, and some ADC drugs that have safety issues caused by excessive dosage may continue to be developed. It can reduce the dosage of ADC drugs, to ensure the safety while ensuring the effectiveness, and significantly improving the possibility of successful ADC drug development. In addition, due to the reduction in the dosage of ADC drug, the production cost is also greatly reduced, which further significantly reduces the economic burden of patients.

### BRIEF DESCRIPTION OF DRAWINGS

FIG. 1 DAR value distribution of Disitamab vedotin detected by HIC-HPLC;

FIG. 2 DAR value distribution of AAJ8D6-ADC detected by HIC-HPLC;

FIG. 3 The effect of single use of Disitamab vedotin and methyl-β-cyclodextrin on tumor cell proliferation;

FIG. 4 The effect of single use of AAJ8D6-ADC and methyl-β-cyclodextrin on tumor cell proliferation.

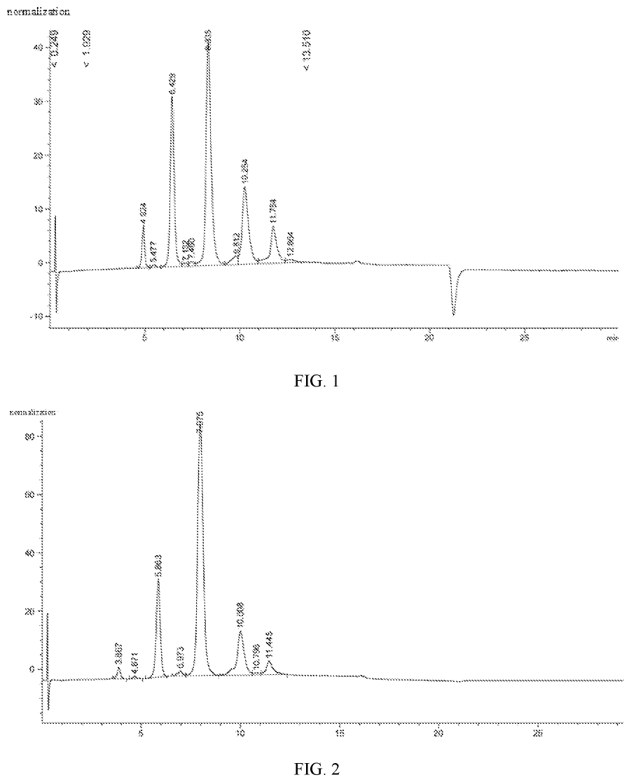

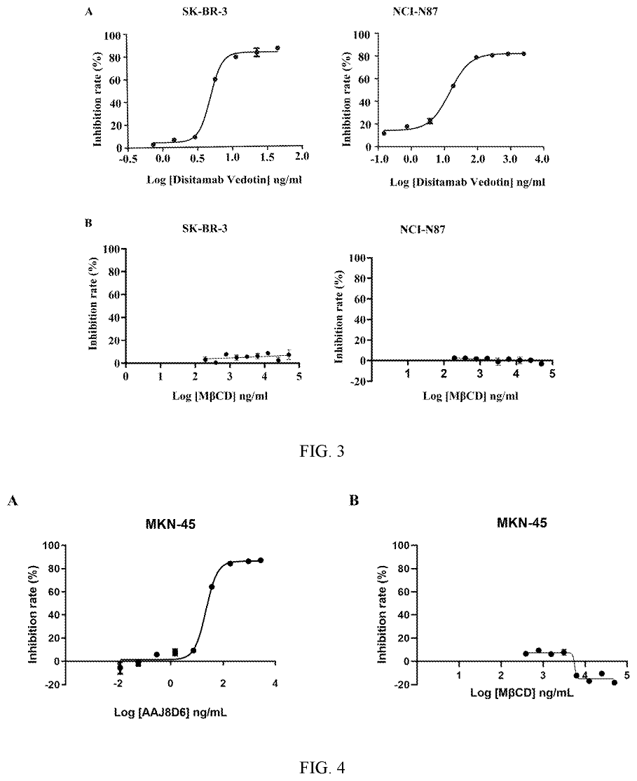

### DETAILED DESCRIPTION

### Definition

Unless otherwise defined, all terms used herein are the same as commonly understood in the art to which the present disclosure belongs. Although any methods and materials similar or equivalent to those described herein can also be used to implement or test the present disclosure, the preferred methods and materials are described herein. In the description and claims of the present disclosure, the following terms are used in accordance with the following definitions.

When a trade name is used in the present disclosure, applicants intend to include the formulation of the trade name product, the generic drug and active drug portion of the trade name product.

Unless stated to the contrary, the terms used in the description and claims have the same meanings as described below.

The term “antibody-drug conjugate” (i.e., ADC) used in the present disclosure refers to a compound in which an antibody or antigen-binding fragment, a linking unit, and an active drug unit are linked together through chemical reaction, and its structure usually consists of three parts: an antibody or antibody-like ligand, a drug moiety (i.e., an active drug unit), and a linker that conjugates the antibody or antibody-like ligand with the drug moiety.

The term “antibody” used in the present disclosure refers to a macromolecular compound that can recognize and bind to antigens or receptors associated with target cells. The function of antibody is to present the drug to the target cell population that binds to the antibody. These antibodies include but not limited to protein hormones, lectins, growth factors, antibodies or other molecules that can bind to cells. In some embodiments, antibodies include murine antibodies, chimeric antibodies, primatized antibodies, humanized antibodies and fully human antibodies (i.e. human antibodies), preferably humanized antibodies and fully human antibodies.

The term “murine antibody” in the present disclosure refers to an antibody prepared in mice. In the preparation, injecting the test subject with specific antigen, and then separating and expressing antibody hybrids with desired sequence or functional properties.

The term “chimeric antibody” refers to an antibody obtained by fusing the variable region of a murine antibody with the constant region of a human antibody, which can reduce the immune response induced by the murine antibody. To build a chimeric antibody, first establishing a hybridoma that secretes murine specific monoclonal antibodies, then cloning the variable region gene from the mouse hybridoma cell, and then cloning the constant region gene of the human antibody as needed. After connecting the mouse variable region gene with the human constant region gene into chimeric gene, inserting the chimeric gene into an expression vector, and finally expressing chimeric antibody molecules in an eukaryotic system or prokaryotic system.

The term “humanized antibody”, also known as CDR-grafted antibody, refers to the transplantation of murine CDR sequences into the framework of human antibody variable region, i.e., antibodies produced in human germline antibody framework sequences of different types. It can overcome the heterologous reaction induced by chimeric antibodies which carry a large amount of murine protein components. Such framework sequences can be obtained from public DNA databases or published references that include germline antibody gene sequences. For example, the germline DNA sequences of human heavy and light chain variable region genes can be found in the “VBase” human germline sequence database (available on the Internet at www.mrccpe.com/ac.uk/vbase), and in Kabat, E.A. et al., 1991 Sequences of Proteins of Immunological Interest, 5th ed. In order to avoid the decrease of activity while the immunogenicity is decreased, the human antibody variable region framework sequence can be subjected to minimal reverse mutation or back mutation to maintain the activity. The humanized antibodies of the present disclosure also include humanized antibodies in which CDRs are further subjected to affinity maturation by phage display. Documents further describing methods involved in humanization using mouse antibodies include, e.g., Queen et al., Proc., Natl. Acad. Sci. USA, 88, 2869, 1991 and the method of Winter and colleagues [Jones., Nature, 321, 522, (1986)], Riechmann, et al. [Nature, 332, 323-327, 1988), Verhoeyen, et al., Science, 239, 1534 (1988)].

The term “fully human antibody”, or “human antibody”, is also known as “fully human monoclonal antibody”, whose variable regions and constant regions are all of human origin, removing immunogenicity and toxic side effects. The development of monoclonal antibodies has gone through four stages, namely: murine monoclonal antibodies, chimeric monoclonal antibodies, humanized monoclonal antibodies and fully human monoclonal antibodies. The present disclosure is a fully human monoclonal antibody. The technologies related to the preparation of fully human antibodies mainly include: human hybridoma technology, EBV transforming B lymphocyte technology, phage display technology, transgenic mouse antibody preparation technology and single B cell antibody preparation technology.

The term “antigen-binding fragment” used in the present disclosure refers to one or more fragments of an antibody that remain the ability to specifically bind to an antigen. Examples of binding fragments contained in “antigen-binding fragments” include (i) Fab fragment, a monovalent fragment consisting of VL, VH, CL and CH1 domains; (ii) F(ab')₂ fragment, a bivalent fragment including two Fab fragments linked by a disulfide bridge, (iii) an Fd fragment consisting of VH and CH1 domains; (iv) an Fv fragment consisting of VH and VL domains of one arm of an antibody; (v) a single domain or dAb fragment (Ward et al., (1989) Nature 341:544-546), which consists of a VH domain; and (vi) isolated complementarity determining regions (CDRs) or (vii) a combination of two or more isolated CDRs may be linked by synthetic linkers. Furthermore, although the two domains VL and VH of the Fv fragment are encoded by separate genes, recombination methods can be used to link them by synthetic linkers, to enable the production of a single protein chain (referred to as single-chain Fv (scFv)), in which the VL and VH domains are paired to form a monovalent molecule; see, e.g., Bird et al. (1988) Science 242:423-426 and Huston et al. (1988) Proc. NatL. Acad. Sci. USA 85:5879-5883). Such single chain antibodies are also intended to be included within the term “antigen-binding fragment” of an antibody. Such antibody fragments are obtained by using conventional technologies in the art, and the fragments are screened for utility in the same manner as that of intact antibodies. Antigen binding moieties can be produced by recombinant DNA technology or by enzymatic or chemical cleavage of intact immunoglobulins. Antibodies can be of different isotypes, e.g., IgG (such as IgG1, IgG2, IgG3, or IgG4 subtypes), IgA1, IgA2, IgD, IgE, or 1 gM antibodies.

The term Fab is an antibody fragment with a molecular weight of about 50,000 and has antigen-binding activity, which is from the fragments obtained by treating IgG antibody molecule with protease papain (which cleaves the amino acid residue at position 224 of the H chain), wherein about half of the N-terminal side of the H chain and the entire L chain are connected together by disulfide bonds.

The term F(ab')₂ is an antibody fragment with a molecular weight of about 100,000 and has antigen-binding activity, including two Fab regions linked at the hinge position, which is obtained by digesting the lower part of two disulfide bonds in the hinge region of IgG with the enzyme pepsin.

The term Fab' is an antibody fragment with a molecular weight of about 50,000 and has antigen-binding activity, which is obtained by cleaving the above-mentioned disulfide bonds of the hinge region of the F(ab')₂. In addition, the Fab' can be produced by inserting the DNA encoding the Fab' fragment of the antibody into a prokaryotic or eukaryotic expression vector and introducing the vector into a prokaryotic or eukaryotic organism to express Fab'.

The term “single-chain construct” includes but is not limited to, “single-chain antibody”, “single-chain Fv” or “scFv”, which is meant to include molecules in which an antibody heavy chain variable domain or region (i.e. VH) is linked with an antibody light chain variable domain or region (i.e. VL) by a linker. Such scFv molecules can have the general structure: NH2-VL-linker-VH-COOH or NH2-VH-linker-VL-COOH. Suitable linkers in prior art consist of repeated GGGGS amino acid sequences or variants thereof, e.g. using 1-4 repeated variants (Holliger et al. (1993), proc. Natl. Acad. Sci. USA 90:6444-6448). Other linkers that may be used in the present disclosure are described by Alfthan et al. (1995), Protein Eng. 8:725-731, Choi et al. (2001), Eur. J. Immuno 1.31:94-106, Hu et al. (1996), Cancer Res. 56:3055-3061, Kipriyanov et al. (1999), J. Mol. Biol. 293:41-56 and Roovers et al. (2001), Cancer Immunol.

Technologies for preparing antibodies or antigen-binding fragments thereof to virtually any target antigen are well known in the art. See, e.g., Kohler and Milstein, Nature 256:495 (1975), and Coligan et al. (eds.), CURRENTPROTOCOLS IN IMMUNOLOGY, Vol. 1, pp. 2.5.1-2.6.7 (John Wiley & Sons, 1991). Briefly, monoclonal antibodies can be obtained by injecting a mouse with a composition including the antigen, taking the spleen to obtain B lymphocytes, fusing the B lymphocytes with myeloma cells to produce hybridomas, cloning the hybridomas, selecting positive clones that produce antibodies to the antigen, culturing the clones that produce antibodies to the antigen, and separating the antibodies from the hybridoma culture. Separation and purification from hybridoma culture can be accomplished by a number of well-established technologies. Such separation technologies include protein A or protein G sepharose affinity chromatography, size exclusion chromatography, and ion exchange chromatography. See, e.g., Coligan, pp. 2.7.1 2.7.12 and pp.2.9.1-2.9.3. See also Baines et al., “Purification of Immunoglobulin G (IgG),” in METHODS IN MOLECULAR BIOLOGY, Vol. 10, pp. 79-104 (The Humana Press, Inc. 1992). After the initial elicitation of antibodies to the immunogen, the antibodies can be sequenced and subsequently prepared by recombinant technologies. Humanization and chimerization of murine antibodies and antibody fragments are known in the art.

The term “linking unit” or “linker” refers to a chemical structural fragment or bond that connects the antibody/antigen-binding fragment at one end and connects the drug at the other end, thus acting as a “bridge” to link the antibody/antigen-binding fragment with the drug molecule. It may include linkers, spacers and amino acid units, and may be synthesized by methods known in the art, such as documented in US2005-0238649A1. As used herein, “linking unit” can be divided into two categories: non-cleavable linkers and cleavable linkers.

Non-cleavable linker is a relatively stable linker whose structure is difficult to degrade and break in vivo. For antibody-drug conjugates containing non-cleavable linkers, the drug release mechanism is as follows: after the conjugate is bound to the antigen and endocytosed by cells, the antibody is enzymatically hydrolyzed in the lysosome to release an active molecule consisting of the drug, linker, and amino acid residues of antibody. The resulting changes in the molecular structure of the drug do not reduce its cytotoxicity, but because the active molecule is charged (amino acid residues), it cannot penetrate into adjacent cells. Therefore, such active drugs cannot kill adjacent tumor cells that do not express the target antigen (antigen-negative cells) (bystander effect) (Bioconjugate Chem. 2010, 21, 5-13). Common linkers such as MC linker and MCC linker, are shown in the following structures.

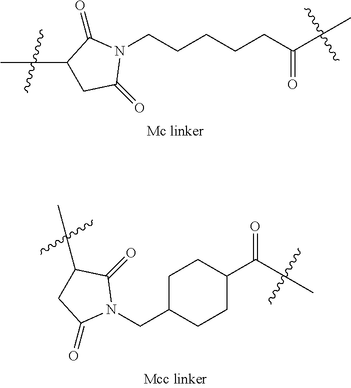 [SMILES: *C(=O)CCCCCN1C(=O)CC(*)C1=O ; *C(=O)C1CCC(CN2C(=O)CC(*)C2=O)CC1]

Cleavable linkers, as the name suggests, can be cleaved within the target cell and release the active drug (the small molecule drug itself). Cleavable linkers can be divided into two main categories: chemically labile linkers and enzymatically labile linkers.

Chemically labile linkers can be selectively cleaved due to differences in plasma and cytoplasmic properties. Such properties include pH, concentration of glutathione, etc.

PH-sensitive linkers are commonly known as acid-cleavable linkers. Such linkers are relatively stable in the neutral environment of blood (pH 7.3-7.5), but will be hydrolyzed in the weakly acidic endosomes (pH 5.0-6.5) and lysosomes (pH 4.5-5.0). Most of the first-generation antibody-drug conjugates use such linkers, such as hydrazones, carbonates, acetals, and ketals. Due to the limited plasma stability of acid-cleavable linkers, antibody-drug conjugates based on such linkers typically have short half-life (2-3 days). This relatively short half-life limits the application of pH-sensitive linkers in new-generation antibody-drug conjugates to an extent.

Glutathione-sensitive linkers are also known as disulfide linkers. Drug release is based on the difference between a high glutathione concentration in the cells (in a millimolar range) and a relatively low glutathione concentration in the blood (in a micromolar range). This is especially true for tumor cells, whose low oxygen content results in enhanced reductase activity and thus a higher glutathione concentration. Disulfide bonds are thermodynamically stable and therefore have good stability in plasma.

Enzymatically labile linkers, such as peptide linkers, allow for better control of drug release. Peptide linkers can be efficiently cleaved by lysosomal proteases such as Cathepsin B or plasmin (whose content is increased in some tumor tissues). Such peptide linkage is thought to be very stable in the plasma circulation because proteases are generally inactive extracellularly due to inappropriate extracellular pH and serum protease inhibitors. Enzymatically labile linkers are widely used as cleavable linkers for antibody-drug conjugates due to their high plasma stability, good intracellular cleavage selectivity and effectiveness.

Suicide linker is typically chimeric between the cleavable linker and the active drug, or suicide linker itself is a part of the cleavable linker. The action mechanism of the suicide linker is that when the cleavable linker is broken under suitable conditions, the suicide linker can spontaneously rearrange its structure, to release the active drug linked to it. Common suicide linkers include p-aminobenzyl alcohols (PAB) and so on.

In some embodiments, the “linking unit” or “linker” can be non-limitingly selected from the following, wherein the wavy line represents the covalent attachment point of the antibody and toxin (drug):

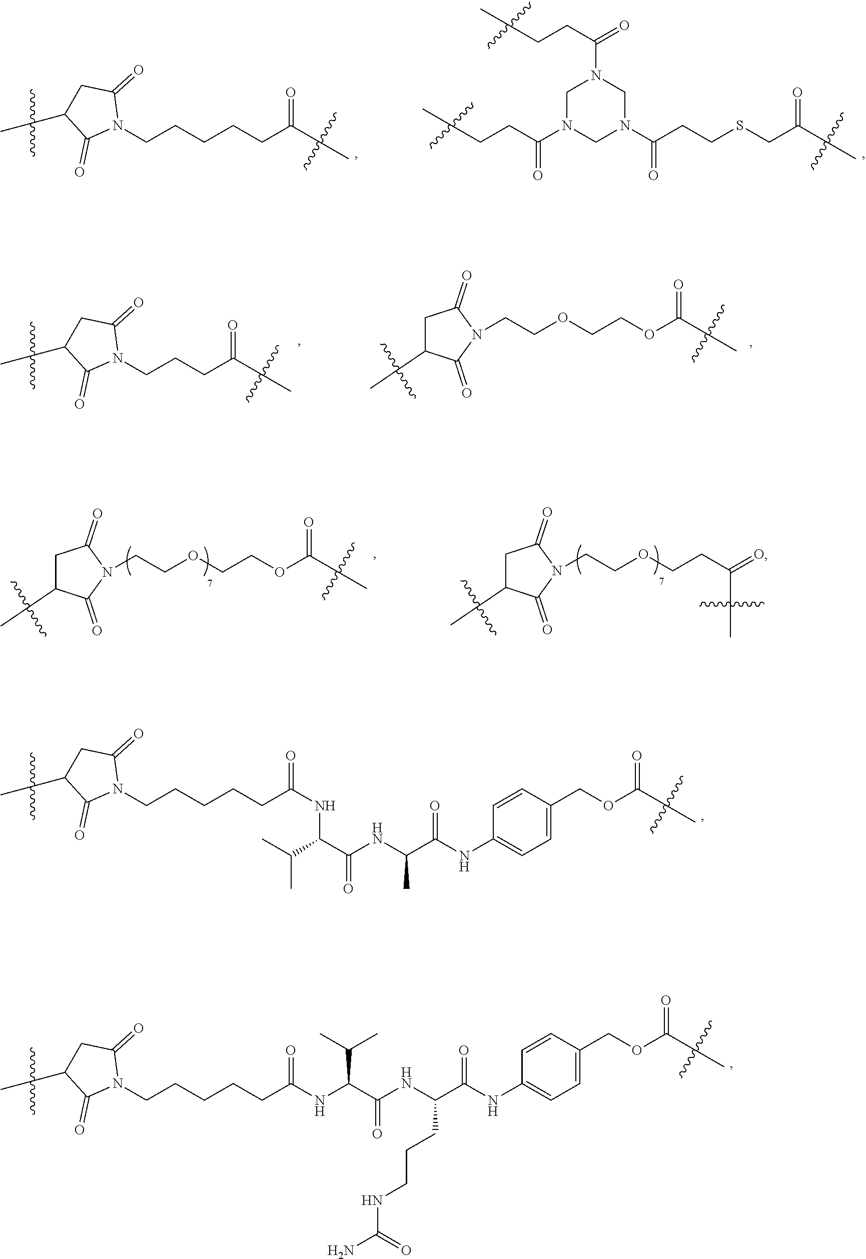

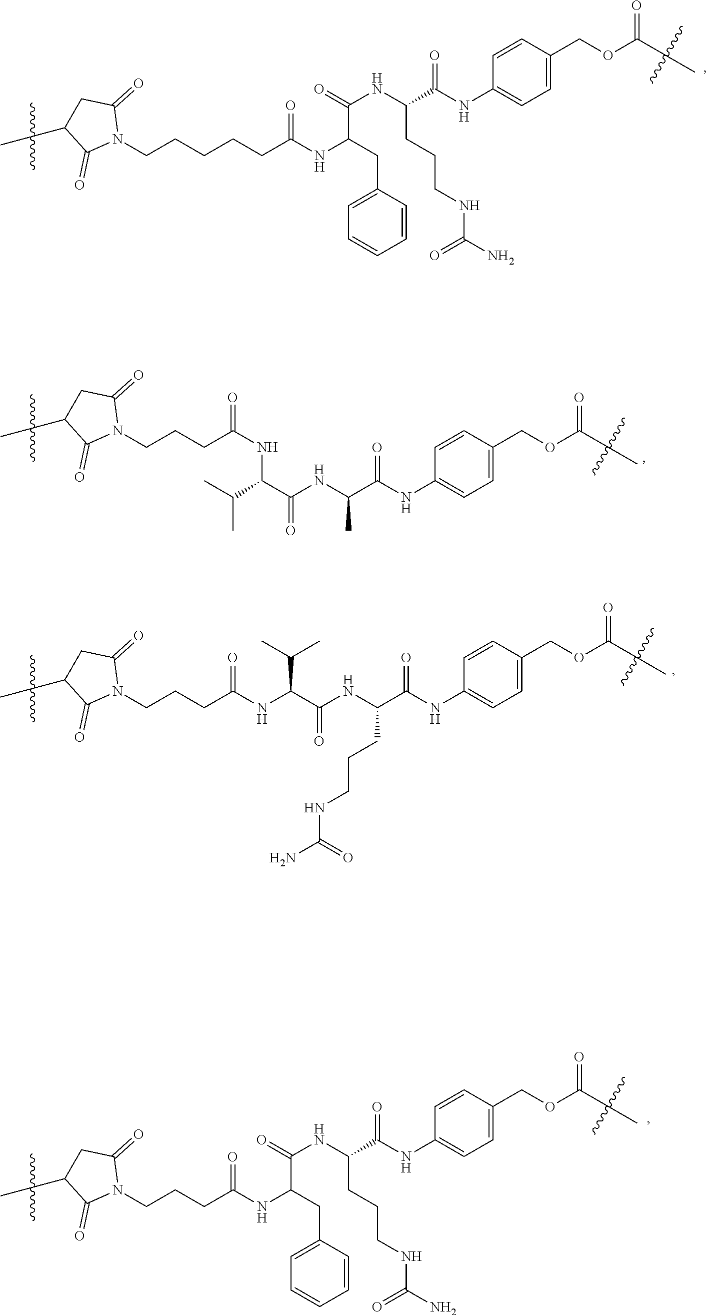 [SMILES: *C(=O)OCc1ccc(NC(=O)[C@H](CCCNC(N)=O)NC(=O)C(Cc2ccccc2)NC(=O)CCCCCN2C(=O)CC(*)C2=O)cc1 ; *C(=O)OCc1ccc(NC(=O)[C@@H](C)NC(=O)[C@@H](NC(=O)CCCN2C(=O)CC(*)C2=O)C(C)C)cc1 ; *C(=O)OCc1ccc(NC(=O)[C@H](CCCNC(N)=O)NC(=O)[C@@H](NC(=O)CCCN2C(=O)CC(*)C2=O)C(C)C)cc1 ; *C(=O)OCc1ccc(NC(=O)[C@H](CCCNC(N)=O)NC(=O)C(Cc2ccccc2)NC(=O)CCCN2C(=O)CC(*)C2=O)cc1]

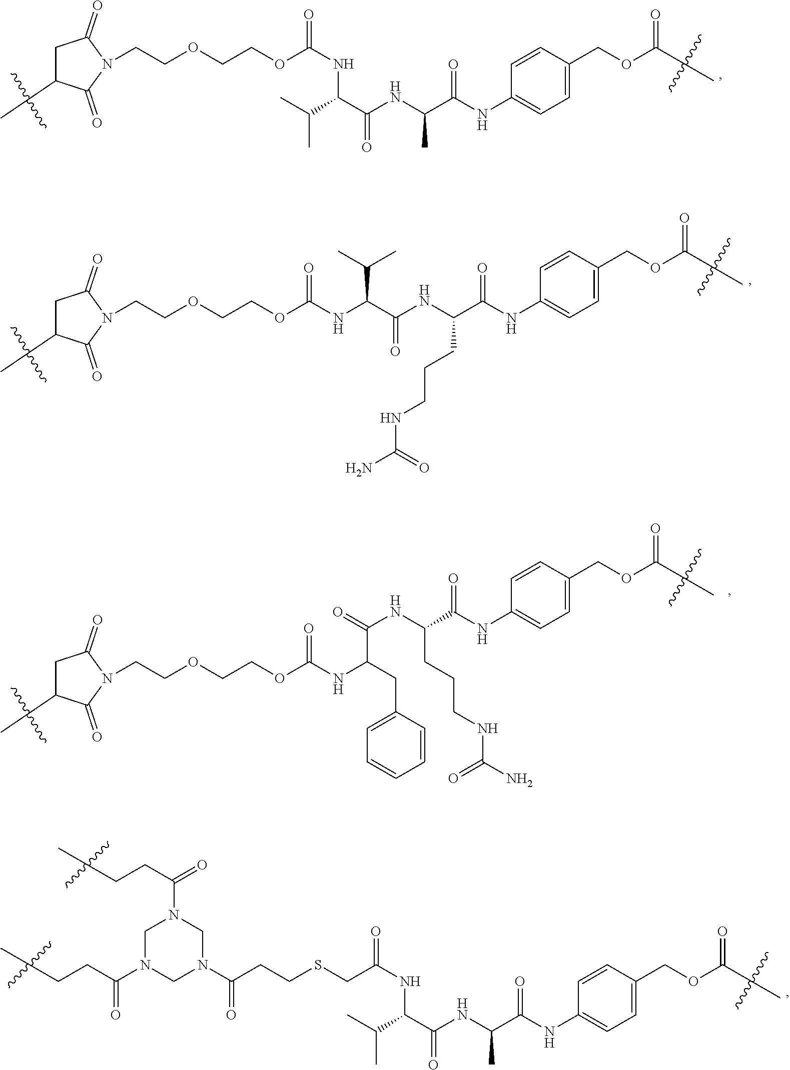 [SMILES: *C(=O)OCc1ccc(NC(=O)[C@@H](C)NC(=O)[C@@H](NC(=O)OCCOCCN2C(=O)CC(*)C2=O)C(C)C)cc1 ; *C(=O)OCc1ccc(NC(=O)[C@H](CCCNC(N)=O)NC(=O)[C@@H](NC(=O)OCCOCCN2C(=O)CC(*)C2=O)C(C)C)cc1 ; *C(=O)OCc1ccc(NC(=O)[C@H](CCCNC(N)=O)NC(=O)C(Cc2ccccc2)NC(=O)OCCOCCN2C(=O)CC(*)C2=O)cc1 ; *CCC(=O)N1CN(C(=O)CC*)CN(C(=O)CCSCC(=O)N[C@H](C(=O)N[C@H](C)C(=O)Nc2ccc(COC(*)=O)cc2)C(C)C)C1]

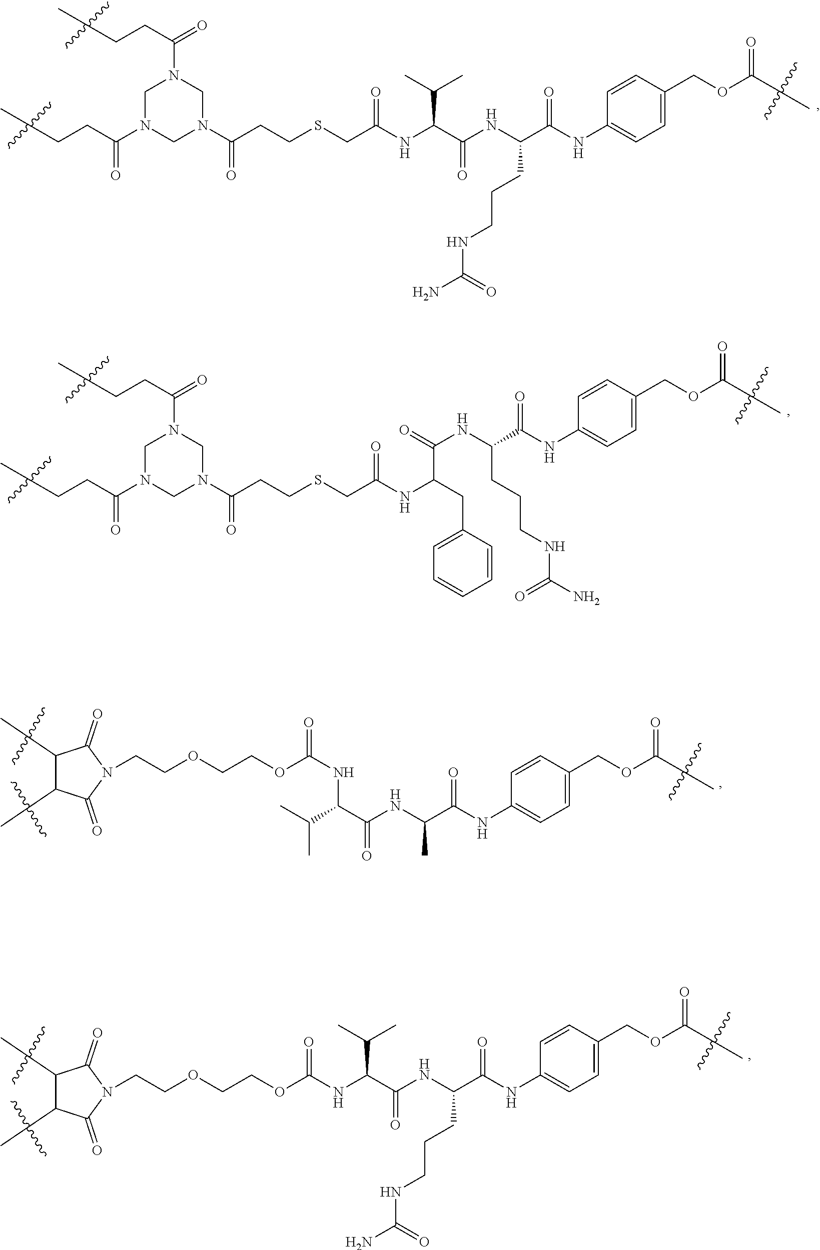 [SMILES: *CCC(=O)N1CN(C(=O)CC*)CN(C(=O)CCSCC(=O)N[C@H](C(=O)N[C@@H](CCCNC(N)=O)C(=O)Nc2ccc(COC(*)=O)cc2)C(C)C)C1 ; *CCC(=O)N1CN(C(=O)CC*)CN(C(=O)CCSCC(=O)NC(Cc2ccccc2)C(=O)N[C@@H](CCCNC(N)=O)C(=O)Nc2ccc(COC(*)=O)cc2)C1 ; *C(=O)OCc1ccc(NC(=O)[C@@H](C)NC(=O)[C@@H](NC(=O)OCCOCCN2C(=O)C(*)C(*)C2=O)C(C)C)cc1 ; *C(=O)OCc1ccc(NC(=O)[C@H](CCCNC(N)=O)NC(=O)[C@@H](NC(=O)OCCOCCN2C(=O)C(*)C(*)C2=O)C(C)C)cc1]

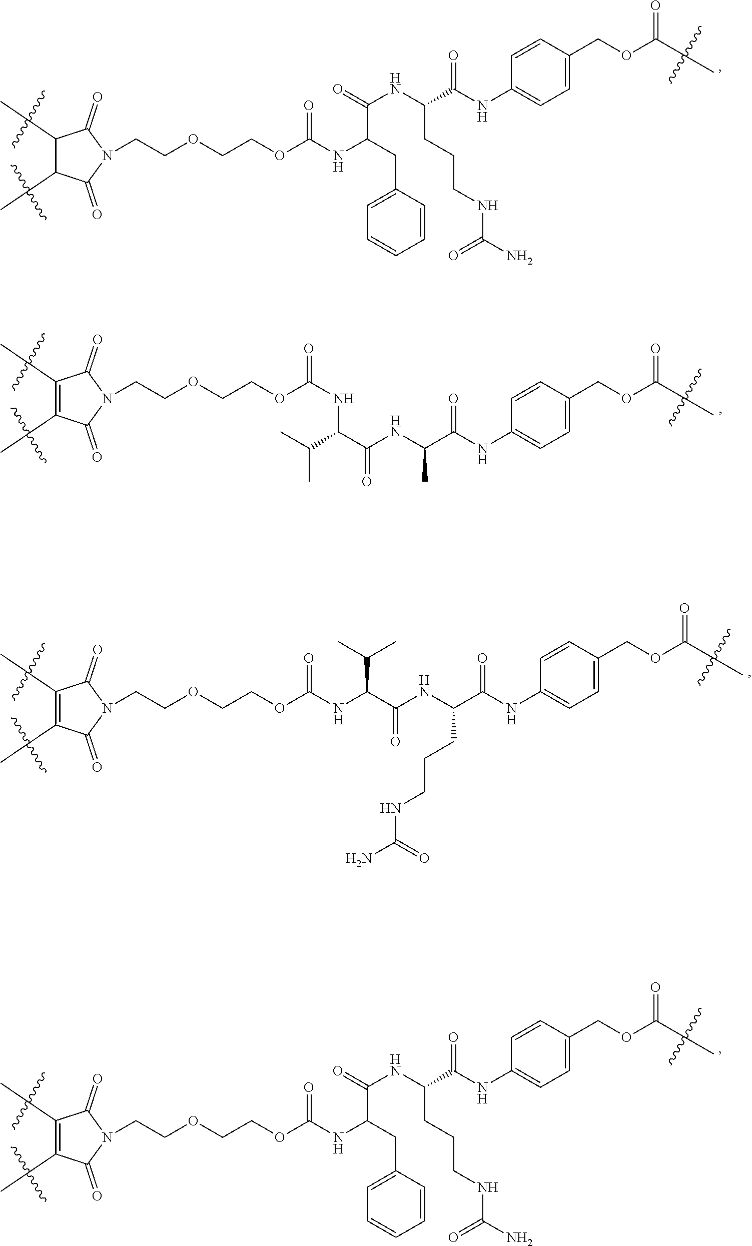 [SMILES: *C(=O)OCc1ccc(NC(=O)[C@H](CCCNC(N)=O)NC(=O)C(Cc2ccccc2)NC(=O)OCCOCCN2C(=O)C(*)C(*)C2=O)cc1 ; *C(=O)OCc1ccc(NC(=O)[C@@H](C)NC(=O)[C@@H](NC(=O)OCCOCCN2C(=O)C(*)=C(*)C2=O)C(C)C)cc1 ; *C(=O)OCc1ccc(NC(=O)[C@H](CCCNC(N)=O)NC(=O)[C@@H](NC(=O)OCCOCCN2C(=O)C(*)=C(*)C2=O)C(C)C)cc1 ; *C(=O)OCc1ccc(NC(=O)[C@H](CCCNC(N)=O)NC(=O)C(Cc2ccccc2)NC(=O)OCCOCCN2C(=O)C(*)=C(*)C2=O)cc1]

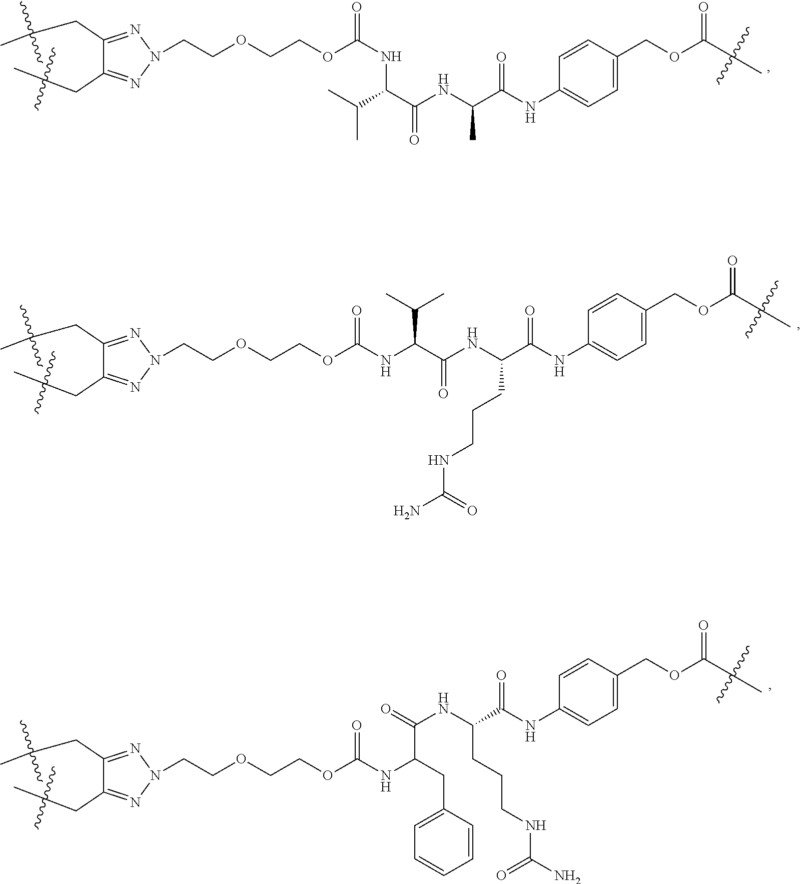 [SMILES: *Cc1nn(CCOCCOC(=O)N[C@H](C(=O)N[C@H](C)C(=O)Nc2ccc(COC(*)=O)cc2)C(C)C)nc1C* ; *Cc1nn(CCOCCOC(=O)N[C@H](C(=O)N[C@@H](CCCNC(N)=O)C(=O)Nc2ccc(COC(*)=O)cc2)C(C)C)nc1C* ; *Cc1nn(CCOCCOC(=O)NC(Cc2ccccc2)C(=O)N[C@@H](CCCNC(N)=O)C(=O)Nc2ccc(COC(*)=O)cc2)nc1C*]

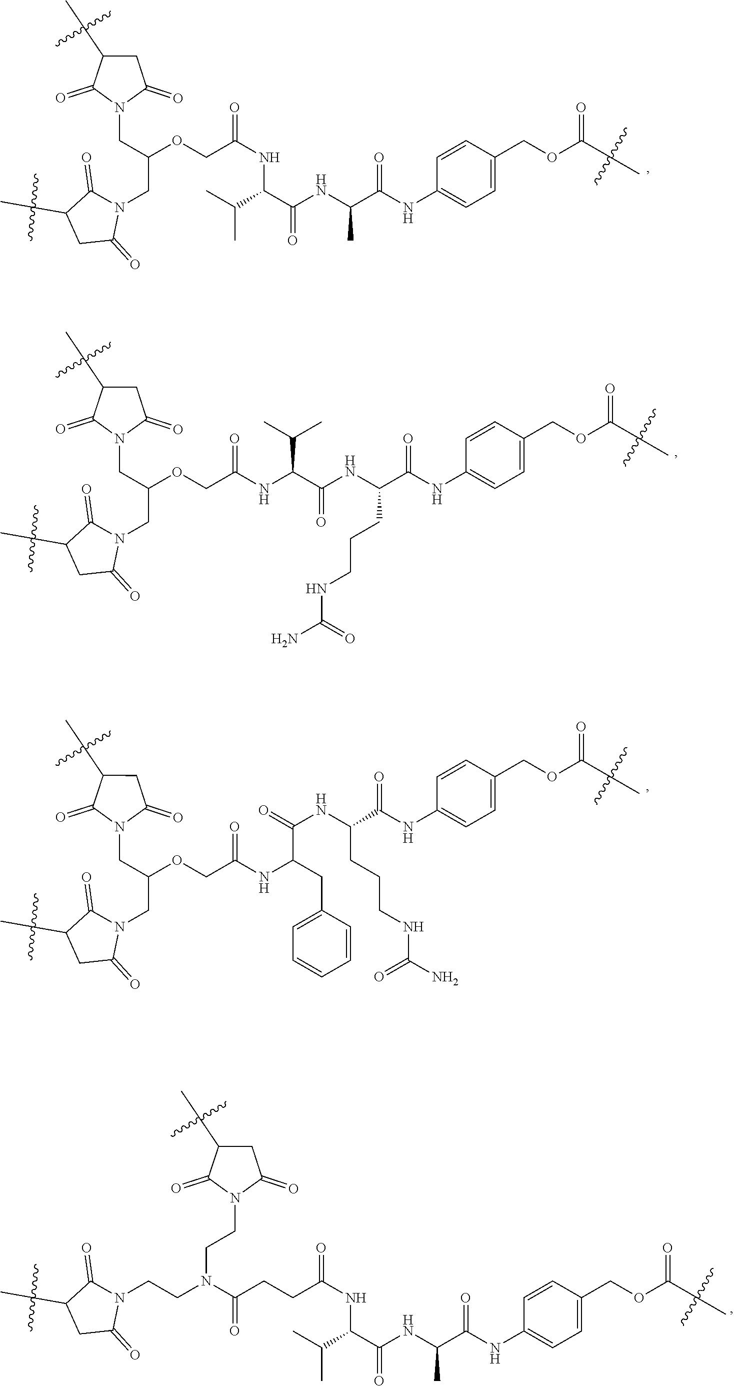 [SMILES: *C(=O)OCc1ccc(NC(=O)[C@@H](C)NC(=O)[C@@H](NC(=O)COC(CN2C(=O)CC(*)C2=O)CN2C(=O)CC(*)C2=O)C(C)C)cc1 ; *C(=O)OCc1ccc(NC(=O)[C@H](CCCNC(N)=O)NC(=O)[C@@H](NC(=O)COC(CN2C(=O)CC(*)C2=O)CN2C(=O)CC(*)C2=O)C(C)C)cc1 ; *C(=O)OCc1ccc(NC(=O)[C@H](CCCNC(N)=O)NC(=O)C(Cc2ccccc2)NC(=O)COC(CN2C(=O)CC(*)C2=O)CN2C(=O)CC(*)C2=O)cc1 ; *C(=O)OCc1ccc(NC(=O)[C@@H](C)NC(=O)[C@@H](NC(=O)CCC(=O)N(CCN2C(=O)CC(*)C2=O)CCN2C(=O)CC(*)C2=O)C(C)C)cc1]

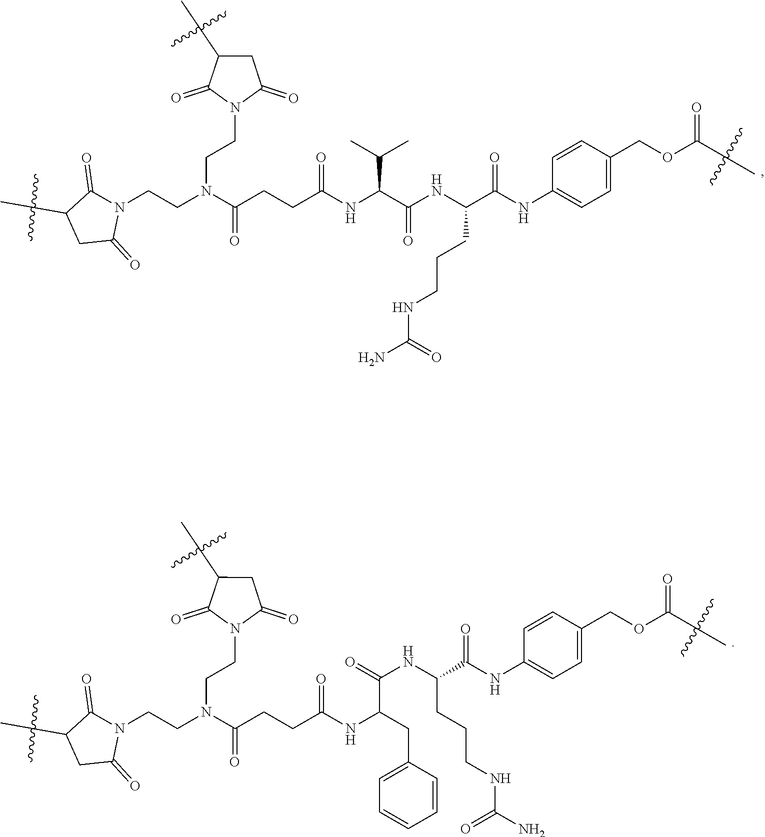 [SMILES: *C(=O)OCc1ccc(NC(=O)[C@H](CCCNC(N)=O)NC(=O)[C@@H](NC(=O)CCC(=O)N(CCN2C(=O)CC(*)C2=O)CCN2C(=O)CC(*)C2=O)C(C)C)cc1 ; *C(=O)OCc1ccc(NC(=O)[C@H](CCCNC(N)=O)NC(=O)C(Cc2ccccc2)NC(=O)CCC(=O)N(CCN2C(=O)CC(*)C2=O)CCN2C(=O)CC(*)C2=O)cc1]

The terms “toxin”, “drug”, “drug moiety” and “drug unit” used in the present disclosure generally refer to the same structure, and can be used in any name in the present disclosure. They generally refer to any compound having the desired biological activity and having reactive functional groups to prepare the conjugates of the present disclosure. Desirable biological activity includes diagnosing, curing, alleviating, treating, preventing disease in humans or other animals. As new drugs are continuously discovered and developed, these new drugs should also be included in the drugs of the present disclosure. It can be any substance that has a deleterious effect on the growth or proliferation of cells, it can be a small molecule toxin and derivatives thereof from bacteria, fungi, plants or animals, and it can include but is not limited to cytotoxic drugs, cell differentiation factors, stem cell nutrition factors, steroid drugs, drugs for the treatment of autoimmune diseases, anti-inflammatory drugs or drugs for the treatment of infectious diseases; or further tubulin inhibitors or DNA damaging agents; or further dolastatin, auristatin, maytansine; calicheamicin, duocarmycin, atramycin derivative PBD, camptothecin derivative SN-38; amanitins, anthracyclines, baccatins, camptothecins, cemadotins, colchicines, colcimids, combretastatins, cryptophycins, discodermolides, docetaxel, doxorubicin, echinomycins, eleutherobins, epothilones, estramustines, lexitropsins, maytansines, methotrexate, netropsins, puromycins, rhizoxins, taxanes, tubulysins, vinca alkaloids, vitamin A precursors, folic acid. In some specific examples, “toxin”, “drug”, “drug moiety”, and “drug unit” can be camptothecin derivatives such as ixatecan, maytansinoids and derivatives thereof (CN101573384) such as DM1, DM3, DM4, auristatin F (AF) and derivatives thereof, such as MMAF, MMAE, 3024 (WO 2016/127790A1), diphtheria toxin, exotoxin, ricin A chain, abrin A chain, modeccin, α-sarcin, Aleutites fordii toxin, dianthin toxin, Phytolaca americana toxin (PAPI, PAPII and PAP-S), Momordica charantia inhibitor, curcin, crotin, sapaonaria officinalis inhibitor, gelonin, mitogellin, restrictocin, phenomycin, enomycin and trichothecenes. In some more specific examples, “toxin”, “drug”, “drug moiety”, and “drug unit” can be non-limitingly selected from the following structures:

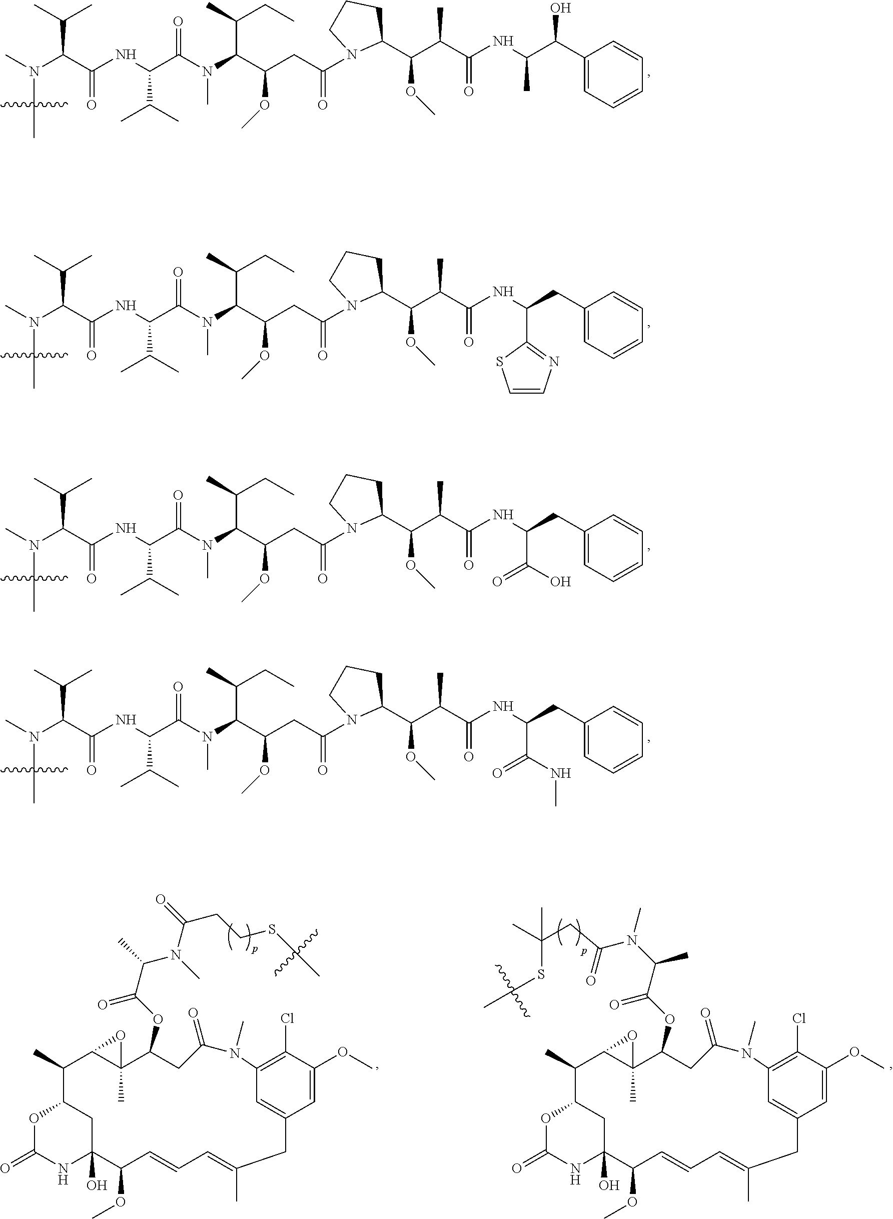

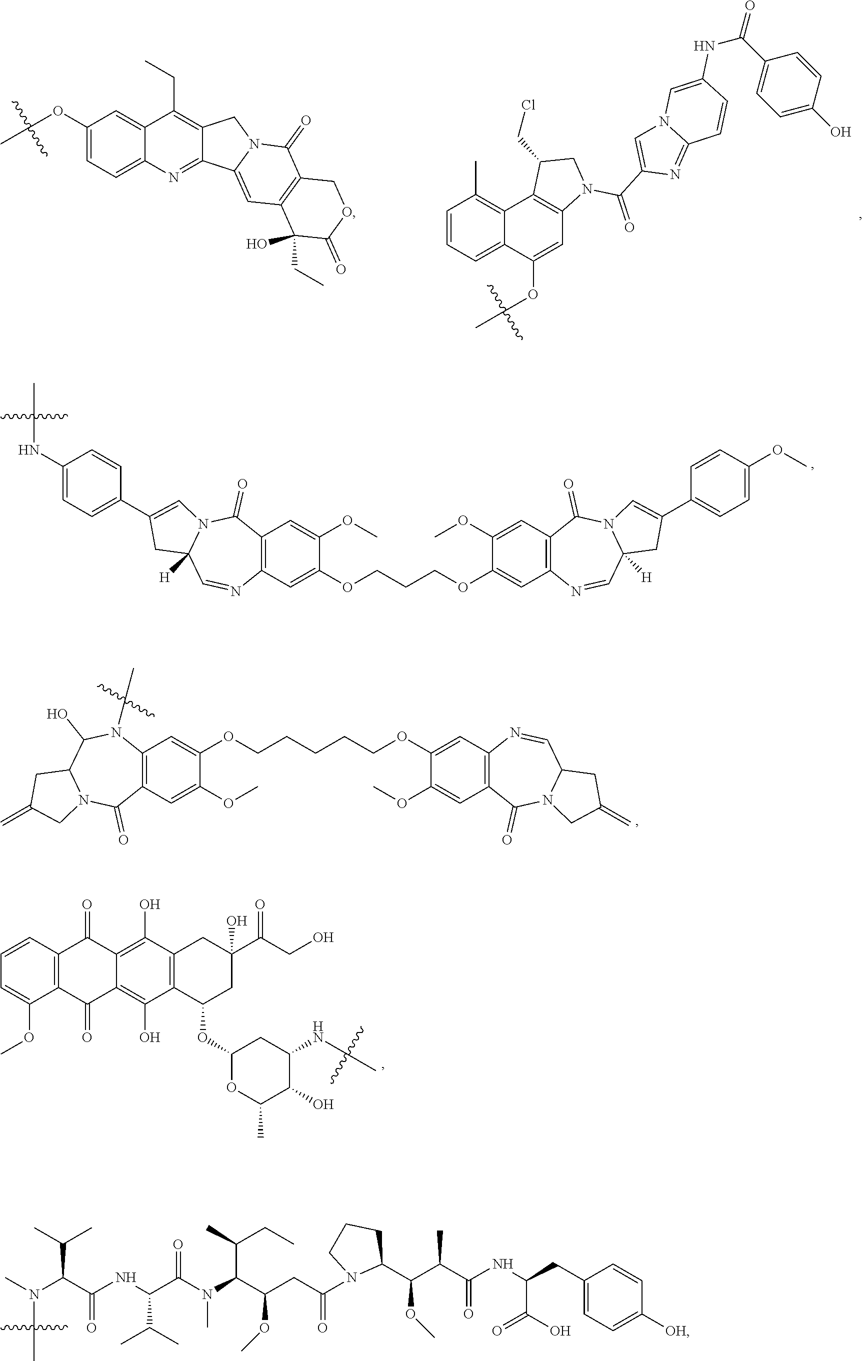

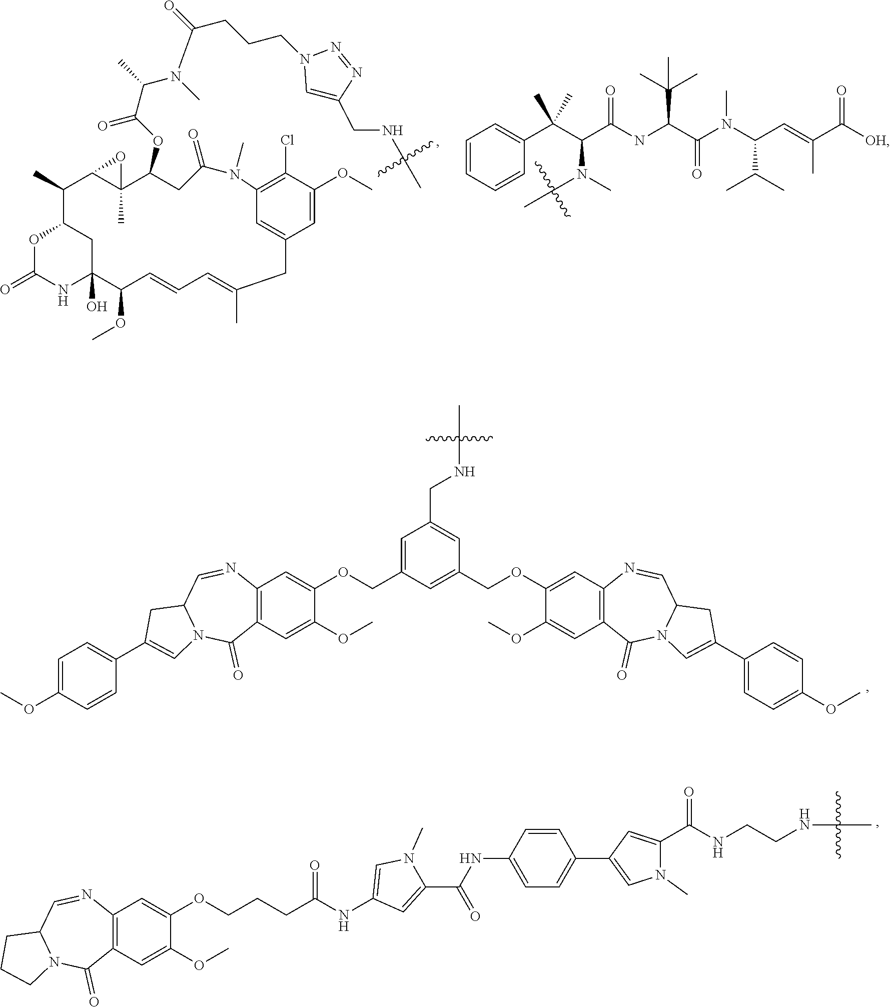

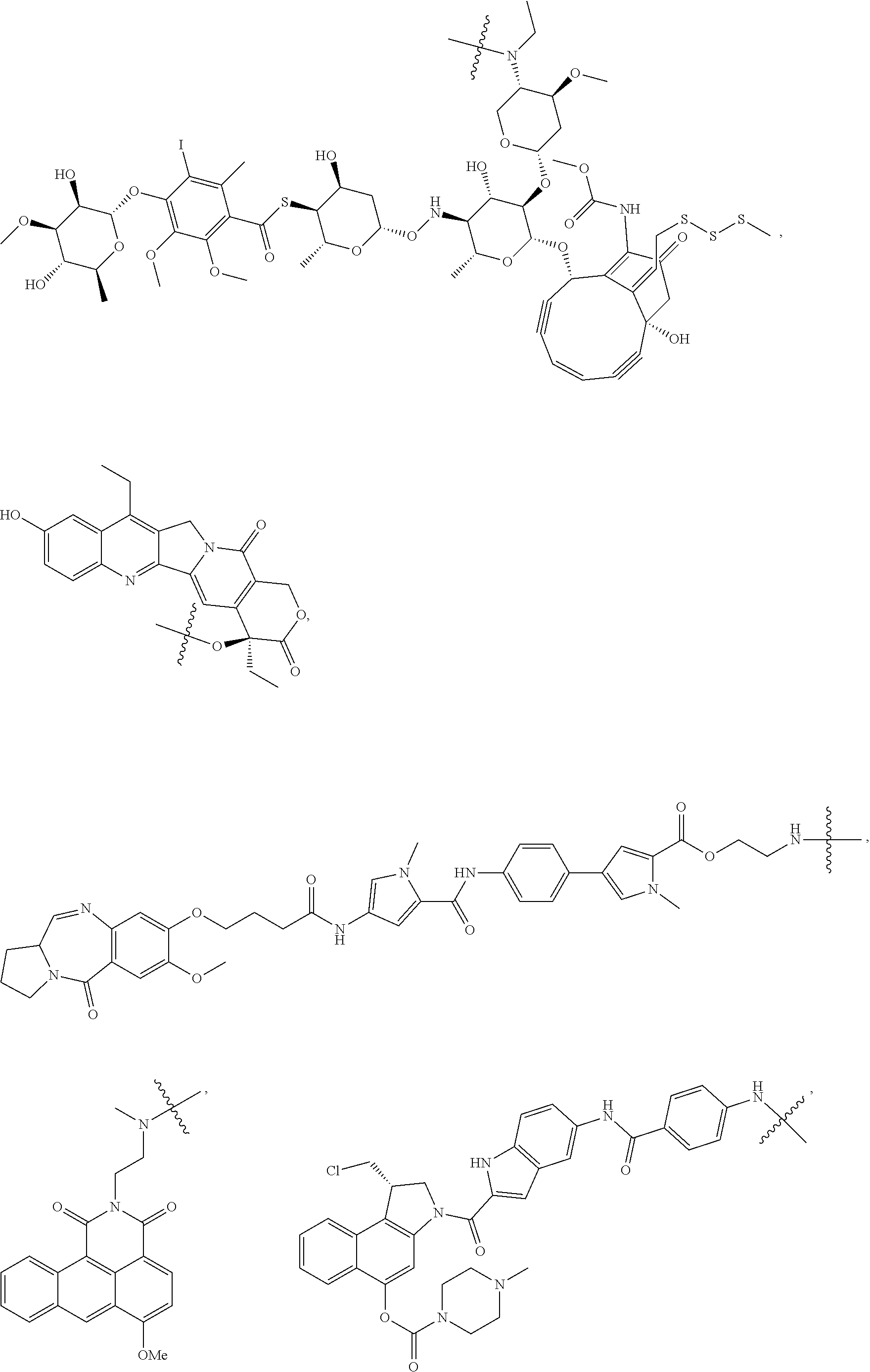

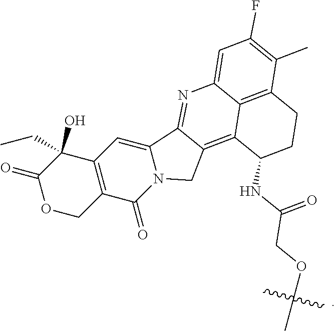 [SMILES: *OCC(=O)N[C@H]1CCc2c(C)c(F)cc3nc4c(c1c23)Cn1c-4cc2c(c1=O)COC(=O)[C@]2(O)CC]

### Specific Examples

The present disclosure will be further elaborated by the following examples. It should be noted that the following examples are intended to further illustrate and explain the present disclosure, and should not be regarded as a limitation of the present disclosure.

### Example 1

### Preparation of Antibody-Drug Conjugates

Antibody-drug conjugates were prepared by a general preparation method:

Method A: The antibody was prepared into a solution of 10 mg/mL with PBS buffer of pH=7.4, added with 2.4 molar equivalents of TCEP, and mixed well by shaking for 1 hour. Then 5.0 molar equivalents of linker-toxin were added, mixed well by shaking to react for 1 h. After the reaction was completed, the residual small molecules were removed by ultrafiltration, and the ultrafilter residue was loaded into hydrophobic interaction chromatography-high performance liquid chromatography (HIC-HPLC) for detection of DAR, drug distribution, and naked antibody ratio.

Method B: The antibody was prepared into a solution of 10 mg/mL with boric acid-borax buffer of pH=9, added with 5.0 molar equivalents of TCEP, and mixed well by shaking for 1 hour. Then 6.0 molar equivalents of linker-toxin were added, mixed well by shaking to react for 3 h. After the reaction was completed, the residual small molecules were removed by ultrafiltration, and the ultrafilter residue was loaded into hydrophobic interaction chromatography-high performance liquid chromatography (HIC-HPLC) for detection of DAR, drug distribution, and naked antibody ratio.

The following two antibody-drug conjugates were prepared by any of the above methods: Disitamab vedotin and AAJ8D6-ADC (i.e. C-Met-Mc-Val-Cit-MMAE, an antibody-drug conjugate targeting C-Met target).

Disitamab vedotin (average DAR 4.01):

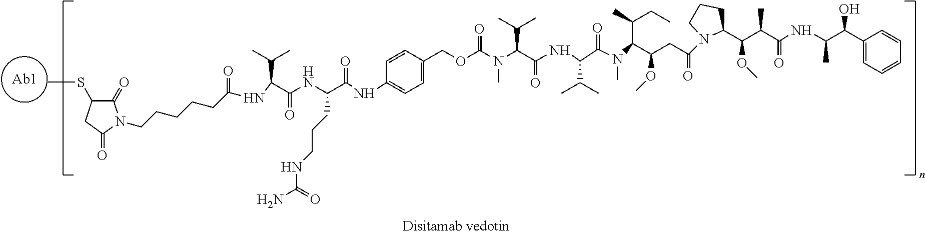

wherein: n represents an integer selected from 1, 2, 3, 4, 5, 6, 7 and 8; Ab1 represents Her2 antibody, and the heavy chain and light chain amino acid sequences thereof are respectively shown as SEQ ID NO: 19 and SEQ ID NO: 20:

| SEQ ID NO: 19 | |
|---|---|
| EVQLVQSGAE VKKPGATVKI SCKVSGYTFT DYYIHWVQQA PGKGLEWMGR | 50 |
| VNPDHGDSYY NQKFKDKATI TADKSTDTAY MELSSLRSED TAVYFCARNY | 100 |
| LFDHWGQGTL VTVSSASTKG PSVFPLAPSS KSTSGGTAAL GCLVKDYFPE | 150 |
| PVTVSWNSGA LTSGVHTFPA VLQSSGLYSL SSVVTVPSSS LGTQTYICNV | 200 |
| NHKPSNTKVD KKVEPKSCDK THTCPPCPAP ELLGGPSVFL FPPKPKDTLM | 250 |
| ISRTPEVTCV VVDVSHEDPE VKENWYVDGV EVHNAKTKPR EEQYNSTYRV | 300 |
| VSVLTVLHQD WLNGKEYKCK VSNKALPAPI EKTISKAKGQ PREPQVYTLP | 350 |
| PSREEMTKNQ VSLTCLVKGF YPSDIAVEWE SNGQPENNYK TTPPVLDSDG | 400 |
| SFFLYSKLTV DKSRWQQGNV FSCSVMHEAL HNHYTQKSLS LSPGK | 445 |
| SEQ ID NO: 20 | |
| DIQMTQSPSS VSASVGDRVT ITCKASQDVG TAVAWYQQKP GKAPKLLIYW | 50 |
| ASIRHTGVPS RFSGSGSGTD FTLTISSLQP EDFATYYCHQ FATYTFGGGT | 100 |
| KVEIKRTVAA PSVFIFPPSD EQLKSGTASV VCLLNNFYPR EAKVQWKVDN | 150 |
| ALQSGNSQES VTEQDSKDST YSLSSTLTLS KADYEKHKVY ACEVTHQGLS | 200 |
| SPVTKSFNRG EC | 212 |

AAJ8D6-ADC (average DAR of 4.02):

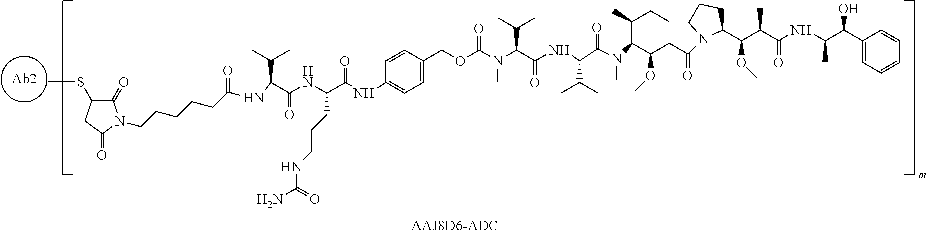

wherein: m represents an integer selected from 1, 2, 3, 4, 5, 6, 7 and 8; Ab2 represents monoclonal antibody targeting C-Met, and the heavy chain and light chain amino acid sequences thereof are respectively shown as SEQ ID NO : 9 and SEQ ID NO: 10:

| SEQ ID NO: 9 | |
|---|---|
| QVQLEQSGPE VVKPGASVKV SCKASGYSFT SYWMHWVRQA PGQGLEWMGM IDPSDSESRL | 60 |
| NQKFKDRVTM TVDTPTSTVY MELSSPRSED TAVYYCARSG YHGTSYWYFD VWGQGTLVTV | 120 |
| SSASTKGPSV FPLAPSSKST SGGTAALGCL VKDYFPEPVT VSWNSGALTS GVHTFPAVLQ | 180 |
| SSGLYSLSSV VTVPSSSLGT QTYICNVNHK PSNTKVDKKV EPKSCDKTHT CPPCPAPELL | 240 |
| GGPSVFLFPP KPKDTLMISR TPEVTCVVVD VSHEDPEVKF NWYVDGVEVH NAKTKPREEQ | 300 |
| YNSTYRVVSV LTVLHQDWLN GKEYKCKVSN KALPAPIEKT ISKAKGQPRE PQVYTLPPSR | 360 |
| EEMTKNQVSL TCLVKGFYPS DIAVEWESNG QPENNYKTTP PVLDSDGSFF LYSKLTVDKS | 420 |
| RWQQGNVFSC SVMHEALHNH YTQKSLSLSP GK | 452 |
| SEQ ID NO: 10 | |
| DIVMTQSPLS LPVTPGEPAS ISCRSSKSLL HSDGITYLYW YLQKPGQSPQ LLIYQMSNLA | 60 |
| SGVPDRFSGS GSGTDFTLKI SRVEAEDVGV YYCAQNLELP PTFGGGTKVE IKRTVAAPSV | 120 |
| FIFPPSDEQL KSGTASVVCL LNNFYPREAK VQWKVDNALQ SGNSQESVTE QDSKDSTYSL | 180 |
| SSTLTLSKAD YEKHKVYACE VTHQGLSSPV TKSENRGEC | 219 |

### Example 2

### In Vitro Anticancer Activity of a Drug Combination of Disitamab Vedotin and Methyl-β-Cyclodextrin on SK-BR-3 Cells

SK-BR-3 cells with a concentration of 5×10⁴/mL were inoculated into 96-well plates at 100 μL/well, and administered according to the use concentration ratio of the combined drug combinations in Table 2. After 72 hours, the cell viability was detected by CCK8 method.

TABLE 2
Combined drug combinations of Disitamab vedotin
and methyl-β-cyclodextrin with different ratios

| | Drug use concentration | Drug use concentration | |
|---|---|---|---|
| Group | of Disitamab vedotin | of methyl-β-cyclodextrin | |
| Combined drug | 4.0 ng/mL | 50000 | ng/mL |
| combination 1-1 | | | |
| Combined drug | 4.0 ng/mL | 25000 | ng/mL |
| combination 1-2 | | | |
| Combined drug | 4.0 ng/mL | 12500 | ng/mL |
| combination 1-3 | | | |
| Combined drug | 4.0 ng/mL | 6250 | ng/mL |
| combination 1-4 | | | |
| Combined drug | 4.0 ng/mL | 3125 | ng/mL |
| combination 1-5 | | | |
| Combined drug | 4.0 ng/mL | 1562.5 | ng/mL |
| combination 1-6 | | | |
| Combined drug | 4.0 ng/mL | 781.25 | ng/mL |
| combination 1-7 | | | |
| Combined drug | 4.0 ng/mL | 390.625 | ng/mL |
| combination 1-8 | | | |

| | | |
|---|---|---|
| Control group 1-1 | 4.0 ng/mL | 0 |

Note:
The drug use concentration refers to the final concentration of the drug.

The Chou-Talalay method (i.e. combination index, CI for short) was used to evaluate the synergistic/antagonistic inhibitory effect of combined action of Disitamab vedotin and methyl-β-cyclodextrin in different ratios for 72 h on SK-BR-3 cell proliferation. The CI values of the combination of the two drugs under different ratio conditions were calculated by using CompuSyn software, further evaluating the combined effect of the two drugs. The evaluation criteria are:

| Range of combination index | Combined effect |
|---|---|
| <0.1 | Very strong synergism |
| 0.1-0.3 | Strong synergism |
| 0.3-0.7 | Synergism |
| 0.7-0.85 | Moderate synergism |
| 0.85-0.90 | Slight synergism |
| 0.90-1.10 | Nearly additive |
| 1.10-1.20 | Slight antagonism |
| 1.20-1.45 | Moderate antagonism |
| 1.45-3.3 | Antagonism |
| 3.3-10 | Strong antagonism |
| >10 | Very strong antagonism |

The results showed that the combined drug combinations of Disitamab vedotin and methyl-β-cyclodextrin with different ratios all had higher inhibitory rates on the proliferation of SK-BR-3 cells than the single drug group, especially the combination groups of high-dose methyl-β-cyclodextrin (312550000 ng/mL) (p<0.05), whose the highest inhibitory rate was (70.79±4.02)%, which had an increase of 1.04 times of that of the single drug group. The CI values indicated that the above combinations were all of synergistic effect (Table 3).

TABLE 3
Effects of combined drug combination on SK-BR-3 cells

| | Inhibitory rate | Combination | |
|---|---|---|---|
| Group | on proliferation | index (CI) | Combined effect |
| Combined drug | 54.99 ± 1.62** | 0.37 | Synergism |
| combination 1-1 | | | |
| Combined drug | 68.44 ± 3.54** | 0.28 | Strong synergism |
| combination 1-2 | | | |
| Combined drug | 70.79 ± 4.02** | 0.27 | Strong synergism |
| combination 1-3 | | | |
| Combined drug | 51.34 ± 2.94** | 0.39 | Synergism |
| combination 1-4 | | | |
| Combined drug | 43.53 ± 1.90* | 0.45 | Synergism |
| combination 1-5 | | | |
| Combined drug | 40.94 ± 3.71 | 0.48 | Synergism |
| combination 1-6 | | | |
| Combined drug | 39.43 ± 4.52 | 0.49 | Synergism |
| combination 1-7 | | | |
| Combined drug | 37.36 ± 1.33 | 0.51 | Synergism |
| combination 1-8 | | | |
| Control group 1-1 | 34.65 ± 0.23 | / | / |

Note:
**p < 0.01;
*p < 0.5

### Example 3

### In Vitro Anticancer Activity of Combined Drug Combinations of Disitamab Vedotin and Methyl-β-Cyclodextrin on NCI-N87 Cells

NCI-N87 cells with a concentration of 5×10⁴/mL were inoculated into 96-well plates at 100 μL/well, and administered according to the use concentration ratios of the combined drug combinations in Table 4. After 72 hours, the cell viability was detected by the CCK8 method.

TABLE 4
Combined drug combinations of Disitamab vedotin
and methyl-β-cyclodextrin in different ratios

| | Drug use concentration | Drug use concentration | |
|---|---|---|---|
| Group | of Disitamab vedotin | of methyl-β-cyclodextrin | |
| Combined drug | 14.0 ng/mL | 50000 | ng/mL |
| combination 2-1 | | | |
| Combined drug | 14.0 ng/mL | 25000 | ng/mL |
| combination 2-2 | | | |
| Combined drug | 14.0 ng/mL | 12500 | ng/mL |
| combination 2-3 | | | |
| Combined drug | 14.0 ng/mL | 6250 | ng/mL |
| combination 2-4 | | | |
| Combined drug | 14.0 ng/mL | 3125 | ng/mL |
| combination 2-5 | | | |
| Combined drug | 14.0 ng/mL | 1562.5 | ng/mL |
| combination 2-6 | | | |
| Combined drug | 14.0 ng/mL | 781.25 | ng/mL |
| combination 2-7 | | | |
| Combined drug | 14.0 ng/mL | 390.625 | ng/mL |
| combination 2-8 | | | |

| | | |
|---|---|---|
| Control group 2-1 | 14.0 ng/mL | 0 |

The Chou-Talalay method (i.e. combination index, CI for short) was used to evaluate the synergistic/antagonistic inhibitory effect of combined action of Disitamab vedotin and methyl-β-cyclodextrin in different ratios for 72 h on NCI-N87 cell proliferation. The CI values of the combination of the two drugs under different ratio conditions were calculated by using CompuSyn software, further evaluating the combined effect of the two drugs. The evaluation criteria are:

| Range of combination index | Combined effect |
|---|---|
| <0.1 | Very strong synergism |
| 0.1-0.3 | Strong synergism |
| 0.3-0.7 | Synergism |
| 0.7-0.85 | Moderate synergism |
| 0.85-0.90 | Slight synergism |
| 0.90-1.10 | Nearly additive |
| 1.10-1.20 | Slight antagonism |
| 1.20-1.45 | Moderate antagonism |
| 1.45-3.3 | Antagonism |
| 3.3-10 | Strong antagonism |
| >10 | Very strong antagonism |

The results showed that the combined drug combinations of Disitamab vedotin and methyl-β-cyclodextrin with different ratios all had higher inhibitory rates on the proliferation of NCI-N87 cells than the single drug group (except for combined drug combination 2-7), especially the combination groups of high-dose methyl-β-cyclodextrin (6250˜50000 ng/mL) had a more significant difference with the single drug group (p<0.05), whose the highest inhibitory rate was (67.16±9.73)%, which had an increase of 56.81% compared with the single drug group (Table 5).

TABLE 5
Effects of combined drug combinations on NCI-N87 cells

| | Inhibitory rate | Combination | |
|---|---|---|---|
| Group | on proliferation | index (CI) | Combined effect |
| Combined drug | 58.81 ± 2.88* | 0.49 | Synergism |
| combination 2-1 | | | |
| Combined drug | 67.16 ± 9.73* | 0.35 | Synergism |
| combination 2-2 | | | |
| Combined drug | 57.10 ± 4.75* | 0.51 | Synergism |
| combination 2-3 | | | |
| Combined drug | 56.74 ± 1.48* | 0.57 | Synergism |
| combination 2-4 | | | |
| Combined drug | 45.66 ± 0.76 | 0.72 | Moderate synergism |
| combination 2-5 | | | |
| Combined drug | 44.61 ± 2.35 | 1.06 | Nearly additive |
| combination 2-6 | | | |
| Combined drug | 40.07 ± 2.74 | 1.18 | Slight antagonism |
| combination 2-7 | | | |
| Combined drug | 47.45 ± 4.49 | 0.70 | Synergism |
| combination 2-8 | | | |
| Control group 2-1 | 42.83 ± 2.21 | / | / |

Note:
**p < 0.01;
*p < 0.5

### Example 4

### In Vitro Anticancer Activity of Combined Drug Combinations of AAJ8D6-ADC and Methyl-β-Cyclodextrin on MKN-45 Cells

MKN-45 cells with a concentration of 5×10⁴/mL were inoculated into 96-well plates at 100 μL/well, and administered according to the use concentration ratios of the combined drug combinations in Table 6. After 72 hours, the cell viability was detected by CCK8 method.

TABLE 6
Combined drug combinations of AAJ8D6-ADC and
methyl-β-cyclodextrin in different ratios

| | Drug use concentration | Drug use concentration | |
|---|---|---|---|
| Group | of AAJ8D6-ADC | of methyl-β-cyclodextrin | |
| Combined drug | 20.0 ng/mL | 50000 | ng/mL |
| combination 3-1 | | | |
| Combined drug | 20.0 ng/mL | 25000 | ng/mL |
| combination 3-2 | | | |
| Combined drug | 20.0 ng/mL | 12500 | ng/mL |
| combination 3-3 | | | |
| Combined drug | 20.0 ng/mL | 6250 | ng/mL |
| combination 3-4 | | | |
| Combined drug | 20.0 ng/mL | 3125 | ng/mL |
| combination 3-5 | | | |
| Combined drug | 20.0 ng/mL | 1562.5 | ng/mL |
| combination 3-6 | | | |
| Combined drug | 20.0 ng/mL | 781.25 | ng/mL |
| combination 3-7 | | | |
| Combined drug | 20.0 ng/mL | 390.625 | ng/mL |
| combination 3-8 | | | |

| | | |
|---|---|---|
| Control group 3-1 | 20.0 ng/mL | 0 |

The Chou-Talalay method (i.e. combination index, CI for short) was used to evaluate the synergistic/antagonistic inhibitory effect of combined action of AAJ8D6-ADC and methyl-β-cyclodextrin in different ratios for 72 h on MKN-45 cell proliferation. The CI values of the combination of the two drugs under different ratio conditions were calculated by using CompuSyn software, further evaluating the combined effect of the two drugs. The evaluation criteria are:

| Range of combination index | Combined effect |
|---|---|
| <0.1 | Very strong synergism |
| 0.1-0.3 | Strong synergism |
| 0.3-0.7 | Synergism |
| 0.7-0.85 | Moderate synergism |
| 0.85-0.90 | Slight synergism |
| 0.90-1.10 | Nearly additive |
| 1.10-1.20 | Slight antagonism |
| 1.20-1.45 | Moderate antagonism |
| 1.45-3.3 | Antagonism |
| 3.3-10 | Strong antagonism |
| >10 | Very strong antagonism |

The results showed that the combined drug combinations of AAJ8D6-ADC and methyl-β-cyclodextrin with different ratios all had higher inhibitory rates on the proliferation of MKN-45 cells than the single drug group, especially the combination groups of high-dose methyl-β-cyclodextrin (3125˜50000 ng/mL) (p<0.05), whose the highest inhibitory rate was (80.85±0.63)%, which had an increase of 61.47% compared with the single drug group. The CI values indicated that the above combinations were all of synergistic effect (Table 7).

TABLE 7
Effects of combined drug combinations on MKN-45 cells

| | Inhibitory rate | Combination | |
|---|---|---|---|
| Group | on proliferation | index (CI) | Combined effect |
| Combined drug | 72.70 ± 2.01** | 0.18 | Strong synergism |
| combination 3-1 | | | |
| Combined drug | 80.85 ± 0.63** | 0.09 | Very strong |
| combination 3-2 | | | synergism |
| Combined drug | 66.90 ± 1.62** | 0.25 | Strong synergism |
| combination 3-3 | | | |
| Combined drug | 67.99 ± 2.09* | 0.24 | Strong synergism |
| combination 3-4 | | | |
| Combined drug | 63.04 ± 2.74** | 0.32 | Synergism |
| combination 3-5 | | | |
| Combined drug | 63.89 ± 5.71* | 0.30 | Synergism |
| combination 3-6 | | | |
| Combined drug | 69.76 ± 6.79* | 0.21 | Strong synergism |
| combination 3-7 | | | |
| Combined drug | 64.15 ± 3.15* | 0.30 | Synergism |
| combination 3-8 | | | |
| Control group3-1 | 50.07 ± 1.68 | / | / |

Note:
**p < 0.01;
*p < 0.5

In this example, two antibody-drug conjugates with different targets, Disitamab vedotin and AAJ8D6-ADC, were used as examples to verify that the combined effect of them with methyl-β-cyclodextrin can enhance the anti-tumor efficacy of antibody-drug conjugate and reduce the dosage of the antibody-drug conjugate in the treatment. It can be understood that the present disclosure only uses these two antibody-drug conjugates as examples to verify the effect, and it should not be regarded as the limitation of targets and specific antibody-drug conjugates.

1. A method of reducing dosage of an antibody-drug conjugate in treatment, comprising using an effective amount of methyl-β-cyclodextrin.

2. The method according to claim 1, wherein molar ratio of methyl-β-cyclodextrin to the antibody-drug conjugate is 200˜40000:0.001˜100; preferably, the molar ratio is 250˜39000:0.01˜10; more preferably, the molar ratio is 300˜38170:0.02˜0.2.

3. The method according to claim 1, wherein the antibody-drug conjugate is used for the treatment of tumors, autoimmune diseases or infectious diseases.

4. The method according to claim 1, wherein the target of the antibody-drug conjugate is selected from BCMA, CD79B, c-Met, GPNMB, IL2RA, LY6E, CD1, CD1a, CD2, CD3, CD4, CD5, CD8, CD11A, CD14, CD15, CD16, CD18, CD19, CD20, CD21, CD22, CD23, CD25, CD29, CD30, CD32b, CD33, CD37, CD38, CD40, CD40L, CD44, CD45, CD46, CD52, CD54, CD55, CD59, CD64, CD67, CD70, CD74, CD79a, CD79b, CD80, CD83, CD95, CD126, CD133, CD138, CD147, CD154, CD166, CD276, HER1, HER2, HER3, MUC1, PTK7, STEAP1, VTCN1, AXL, BCMA, CA9, CASP, CASP3, CDH3, CDKs, CEACAM5, CLDN18, c-Met, Cripto-1, CTL4, DLL3, EF2, EFNA4, EGFR, ENPP3, EphA2, ETBR, FGFR2, FGFR3, FOLR1, FOLR1, Ganglioside, GCPII, HER2, HER3, HGFR, HLA-DR, IGF1R, IL3RA, ITGAV, ITGB3, KIT, LAMP1, Lewis-Y, LRRC15, LY75, LYPD3, MCP, MELTF, MSLN, MUC1, MUC16, NaPi-2b, NCAM1, NECTIN4, NOTCH3, Prolactin receptor, RNA polymerase II, ROR1, SDC1, SGLT2, SLAMF6, SLAMF7, SLITRK6, STAR, STING, TfR, TIM1, TLR8, TNF, TOP1, TPBG, Trop-2, VEGF , ZIP6, cytokines, tubulins and combinations thereof; preferably, the target of the antibody-drug conjugate is selected from CD19, EGFR, BCMA, Trop-2, TOP1, NECTIN4, CD79B, CD22, HER2, CD30, CD33, c-Met, cytokines, tubulins and combinations thereof.

5. The method according to claim 1, wherein the antibody-drug conjugate is selected from Loncastuximab tesirine, Cetuximab sarotalocan, Belantamab mafodotin, Sacituzumab govitecan, Fam-trastuzumab deruxtecan, Enfortumab vedotin, Polatuzumab vedotin, Inotuzumab ozogamicin, Ado-trastuzumab emtansine, Brentuximab vedotin, Gemtuzumab ozogamicin, Disitamab vedotin, Tisotumab vedotin, Depatuxizumab mafodotin, TAA-013, Trastuzumab duocarmazine, KSI-301, BAT-8001, Rovalpituzumab tesirine, SAR-408701, datopotamab, Mirvetuximab soravtansine, ARX-788, Trastuzumab emtansine, Telisotuzumab vedotin, SHR-A1403.

6. The method according to claim 1, wherein the tumor is solid tumor or non-solid tumor; preferably, the tumor is selected from hematopoietic tumor, carcinoma, sarcoma, melanoma and glial tumor; more preferably, the tumor is selected from solid tumors and blood tumors such as breast cancer, ovarian cancer, cervical cancer, uterine cancer, prostate cancer, kidney cancer, urethral cancer, bladder cancer, liver cancer, stomach cancer, endometrial cancer, salivary gland cancer, esophagus cancer, lung cancer, colon cancer, rectal cancer, colorectal cancer, bone cancer, skin cancer, thyroid cancer, pancreatic cancer, melanoma, glioma, neuroblastoma, glioblastoma multiforme, sarcoma, lymphoma, leukemia; the autoimmune disease is selected from immune-mediated thrombocytopenia, dermatomyositis, Sjogren's syndrome, multiple sclerosis, Siddenham's chorea, myasthenia gravis, systemic lupus erythematosus, lupus nephritis, rheumatic fever, rheumatoid arthritis, polyglandular syndrome, bullous pemphigoid, diabetes, Hen-scherer's purpura, post-streptococcal nephritis, erythema nodosum, Takayasu's arteritis, Addison's disease, sarcoidosis, ulcerative colitis, erythema multiforme, IgA nephropathy, polyarteritis nodosa, ankylosing spondylitis, Goodpas' Hill syndrome, thromboangiitis obliterans, primary biliary cirrhosis, Hashimoto's thyroiditis, thyrotoxicosis, scleroderma, chronic active hepatitis, polymyositis/dermatomyositis, polychondritis, pemphigus vulgaris, Wegener's granulomatosis, membranous nephropathy, amyotrophic lateral sclerosis, tabes, giant cell arteritis/polymyalgia, pernicious anemia, rapidly progressive glomerulonephritis, fibrotic alveolitis, and juvenile diabetes and emerging diseases;
the infectious disease is human immunodeficiency virus (HIV), Mycobacterium tuberculosis, Streptococcus agalactiae, Methicillin-resistant Staphylococcus aureus, Legionella pneumophila, Streptococcus pyogenes, Escherichia coli, Neisseria gonorrhoeae, Neisseria meningitidis, Pneumococcus, Haemophilus influenzae type B, Treponema pallidus, Lyme disease Treponema, West Nile virus, Pseudomonas aeruginosa, Mycobacterium leprae, Bacillus abortus, rabies virus, influenza virus, cytomegalovirus, herpes simplex virus type I, herpes simplex virus type II, human serum parvovirus, respiratory syncytial virus, varicella-zoster virus, Hepatitis B virus, measles virus, adenovirus, human T-cell leukemia virus, Epstein-Barr virus, murine leukemia virus, mumps virus, vesicular stomatitis virus, Sindbis virus, lymphocytic choriomeningitis virus, wart virus, bluetongue virus, Sendai virus, feline leukemia virus, reovirus, polio virus, simian virus 40, murine mammary tumor virus, dengue virus, rubella virus, Plasmodium falciparum, Plasmodium vivax , Toxoplasma gondii, Trypanosoma johnsonii, Trypanosoma cruzi, Trypanosoma rhodesiana, Trypanosoma brucei, Schistosoma mansonii, Schistosoma japonicum, Babesia bovis, Eimeria tenella, Onchocerciasis, tropical Leishmania, Trichinella spiralis, Pyremys vulgaris, Taenia vesicularis, Taenia lamblia, Taenia saginata, Echinococcus granulosus, Taenia mesogenes, Mycoplasma arthritis, Mycoplasma hyorrhis, Mycoplasma oralis, Mycoplasma pyogenes, Acholesteria reinhardtii, Mycoplasma salivarius and Mycoplasma pneumoniae and emerging diseases.

7. The method according to claim 1, wherein methyl-β-cyclodextrin is used as one of the excipient components of an antibody-drug conjugate preparation.

8. The method according to claim 1, wherein methyl-β-cyclodextrin is developed into a methyl-β-cyclodextrin preparation and used in combination with an antibody-drug conjugate.

9. A method of reducing therapeutic dosage of an antibody-drug conjugate, comprising using methyl-β-cyclodextrin.

10. An antibody-drug conjugate preparation, comprising an effective amount of methyl-β-cyclodextrin as an excipient.

11. The pharmaceutical composition according to claim 10, wherein molar ratio of methyl-β-cyclodextrin to the antibody-drug conjugate is 200˜40000:0.001˜100; preferably, the molar ratio is 250˜39000:0.01˜10; more preferably, the molar ratio is 300˜38170:0.02˜0.2.

12. A method for treating diseases with combined drug combination, wherein the drug combination comprises: an effective dosage of methyl-β-cyclodextrin or a pharmaceutically acceptable excipient thereof, and an antibody-drug conjugate or a pharmaceutically acceptable excipient thereof.

13. The method according to claim 12, wherein molar ratio of methyl-β-cyclodextrin to the antibody-drug conjugate is 200˜40000:0.001˜100; preferably, the molar ratio is 250˜39000:0.01˜10; more preferably, the molar ratio is 300˜38170:0.02˜0.2.


SEQUENCE LISTING (20 sequences)

SEQ ID NO: 1 - protein, 5 aa, artificial sequence
  heavy chain variable region CDR1
SYWMH

SEQ ID NO: 2 - protein, 17 aa, artificial sequence
  heavy chain variable region CDR2
MIDPSDSESRLNQKFKD

SEQ ID NO: 3 - protein, 13 aa, artificial sequence
  heavy chain variable region CDR3
SGYHGTSYWYFDV

SEQ ID NO: 4 - protein, 16 aa, artificial sequence
  light chain variable region CDR1
RSSKSLLHSDGITYLY

SEQ ID NO: 5 - protein, 7 aa, artificial sequence
  light chain variable region CDR2
QMSNLAS

SEQ ID NO: 6 - protein, 9 aa, artificial sequence
  light chain variable region CDR3
AQNLELPPT

SEQ ID NO: 7 - protein, 122 aa, artificial sequence
  heavy chain variable region
QVQLEQSGPEVVKPGASVKVSCKASGYSFTSYWMHWVRQAPGQGLEWMGMIDPSDSESRLNQKFKDRVTMTVDTPTSTVYMELSSPRSEDTAVYYCARSGYHGTSYWYFDVWGQGTLVTVSS

SEQ ID NO: 8 - protein, 115 aa, artificial sequence
  light chain variable region
DIVMTQSPLSLPVTPGEPASISCRSSKSLLHSDGITYLYWYLQKPGQSPQLLIYQMSNLASGVPDRFSGSGSGTDFTLKISRVEAEDVGVYYCAQNLELPPTFGGGTKVEIKRTV

SEQ ID NO: 9 - protein, 452 aa, artificial sequence
  heavy chain
QVQLEQSGPEVVKPGASVKVSCKASGYSFTSYWMHWVRQAPGQGLEWMGMIDPSDSESRLNQKFKDRVTMTVDTPTSTVYMELSSPRSEDTAVYYCARSGYHGTSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 219 aa, artificial sequence
  light chain
DIVMTQSPLSLPVTPGEPASISCRSSKSLLHSDGITYLYWYLQKPGQSPQLLIYQMSNLASGVPDRFSGSGSGTDFTLKISRVEAEDVGVYYCAQNLELPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 8 aa, artificial sequence
  heavy chain variable region CDR1
GYTFTDYY

SEQ ID NO: 12 - protein, 8 aa, artificial sequence
  heavy chain variable region CDR2
VNPDHGDS

SEQ ID NO: 13 - protein, 8 aa, artificial sequence
  heavy chain variable region CDR3
ARNYLFDH

SEQ ID NO: 14 - protein, 6 aa, artificial sequence
  light chain variable region CDR1
QDVGTA

SEQ ID NO: 15 - protein, 3 aa, artificial sequence
  light chain variable region CDR2
WAS

SEQ ID NO: 16 - protein, 7 aa, artificial sequence
  light chain variable region CDR3
HQFATYT

SEQ ID NO: 17 - protein, 115 aa, artificial sequence
  heavy chain variable region
EVQLVQSGAEVKKPGATVKISCKVSGYTFTDYYIHWVQQAPGKGLEWMGRVNPDHGDSYYNQKFKDKATITADKSTDTAYMELSSLRSEDTAVYFCARNYLFDHWGQGTLVTVSS

SEQ ID NO: 18 - protein, 108 aa, artificial sequence
  light chain variable region
DIQMTQSPSSVSASVGDRVTITCKASQDVGTAVAWYQQKPGKAPKLLIYWASIRHTGVPSRFSGSGSGTDFTLTISSLQPEDFATYYCHQFATYTFGGGTKVEIKRTV

SEQ ID NO: 19 - protein, 445 aa, artificial sequence
  heavy chain
EVQLVQSGAEVKKPGATVKISCKVSGYTFTDYYIHWVQQAPGKGLEWMGRVNPDHGDSYYNQKFKDKATITADKSTDTAYMELSSLRSEDTAVYFCARNYLFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 212 aa, artificial sequence
  light chain
DIQMTQSPSSVSASVGDRVTITCKASQDVGTAVAWYQQKPGKAPKLLIYWASIRHTGVPSRFSGSGSGTDFTLTISSLQPEDFATYYCHQFATYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC